(19) United States
(12) Patent Application Publication
(10) Pub. No.: US 2026/0226447 A1
(43) Pub. Date: Aug. 6, 2026

(54) MODIFIED SUBTILISIN PROTEINS & USES THEREOF

(71) Applicant: University of Tennessee Research Foundation, Knoxville, TN (US)

(72) Inventors: Jordan Cannon, Knoxville, TN (US); Todd Reynolds, Knoxville, TN (US)

(73) Assignee: University of Tennessee Research Foundation, Knoxville, TN (US)

(21) Appl. No.: 19/045,921

(22) Filed: Feb. 5, 2025

Related U.S. Application Data
(63) Continuation of application No. PCT/US2023/071699, filed on Aug. 4, 2023.
(60) Provisional application No. 63/370,547, filed on Aug. 5, 2022.
(60) Provisional application No. 63/471,109, filed on Jun. 5, 2023.

(51) Int. Cl. C12N 9/64; C08J 11/10; C12N 9/02; C12N 9/04

(52) U.S. Cl. CPC C12N 9/6408; C08J 11/105; C12N 9/0006; C12N 9/0051; C12Y 101/01027; C12Y 108/01004; C12Y 304/21062

(57) ABSTRACT

Modified subtilisin proteins and polypeptides are provided herein. Many of the modified subtilisin proteins and polypeptides exhibit an altered poly(L) lactic acid (PLLA) depolymerizing activity. Methods of depolymerizing PLLA methods of producing lactic acid, lactide and lactones from PLLA and methods of recycling plastic materials comprising PLLA with a modified subtilisin polypeptide are provided herein.

### CROSS-REFERENCE TO RELATED APPLICATIONS

This application is a continuation application of PCT/US2023/071699, filed Aug. 4, 2023, which claims priority to U.S. Provisional Application No. 63/471,109, filed Jun. 5, 2023, and U.S. Provisional Application No. 63/370,547, filed Aug. 5, 2022; each of which is incorporated by reference in its entirety.

### SEQUENCE LISTING INCORPORATION BY REFERENCE

The official copy of the Sequence Listing is submitted concurrently with the specification as an WIPO Standard ST.26 formatted XML file with file name “5820.047WO1.xml”, a creation date of Jul. 29, 2023, and a size of 123 kilobytes. This Sequence Listing filed via USPTO Patent Center is part of the specification and is incorporated in its entirety by reference herein.

### TECHNICAL FIELD

Disclosed are compositions and methods relating to Bacillus subtilisin variants. The subtilisin variants are useful, for example, in depolymerizing poly-L-lactic acid (PLLA). The variants, methods, and systems described herein provide depolymerization of PLLA into lactate, potentially for re-use in new poly-L-lactic acid polymers.

### INCORPORATION BY REFERENCE

All references cited herein, including patents, patent applications and publications, are incorporated by reference in their entireties to the same extent as if each individual publication, patent, or patent application was specifically and individually indicated to be incorporated by reference.

### BACKGROUND OF THE INVENTION

Global annual plastic production has increased ^{~}38% from 265 million metric tons in 2010 to 367 million metric tons in 2020^{1,2}. The overwhelming majority of these plastics are generated from the polymerization of non-renewable, fossil fuel-based hydrocarbons that are non-biodegradable³. Given the environmental issues stemming from non-biodegradability of these plastics⁴⁻⁶, there has been increased interest in eco-friendly, biodegradable plastics to ameliorate plastic waste issues and improve sustainability⁷. Biodegradable plastics are created from either bio- or petroleum-based resources⁷. The biodegradability of these plastics is the result of specific functional groups, e.g., esters, within the polymer backbone that microbial enzymes can recognize and degrade. Biobased, biodegradable plastics provide an ideal alternative to recalcitrant, petroleum-based plastics as they create a sustainable carbon cycle in which renewable natural sources (e.g., plant materials) are used to create the plastics⁸ and these same natural sources consume the CO₂ that results from plastic biodegradation⁹⁻¹¹. Despite apparent sustainability relative to petroleum-based plastics, lack of infrastructure for biodegradable plastic production and recycling in combination with low biodegradability outside of controlled conditions creates a barrier for their implementation^{10,12}. The vast majority of these plastics are used in packaging, and current projections indicate that bioplastic production will increase 300% by 2026¹³. Given the single-use nature of packaging materials, investigation of viable disposal and recycling options for these plastics becomes essential to ensure they do not accumulate as alternative plastic waste products. This holds especially true for poly-L-lactic acid (PLLA) which has limited biodegradability relative to other biodegradable plastics^{14, 15}.

PLLA is one of the most promising candidate to replace non-biodegradable, petroleum-based plastics as the source material (L-lactic acid) is made from the fermentation of renewable starchy and cellulosic plant materials¹⁵. PLLA has physical properties that are similar to non-biodegradable plastics, i.e., polystyrene and polyethylene terephthalate^{17,18}, giving it broad applicability in things such as packaging^{9,13}, 3D printing, and in biodegradable agricultural mulches^{16,19}. Most importantly, this plastic is 100% biodegradable with complete mineralization occurring under industrial composing conditions^{10,20}. However, outside of tightly controlled composting conditions, PLLA biodegrades very slowly¹⁰. Considering end-of-life options for PLLA is important to ensure it does not become an alternative environmental pollutant. In particular, this should be considered holistically with regard to creating a circular economy for PLLA in which products are made and continually recycled²¹. Notably, PLLA recycling is more energetically favorable than de novo production of L-lactate monomers by glucose fermentation²².

The vast majority of currently identified PLLA depolymerizing enzymes are serine proteases produced by Firmicutes^{26,30-33} and Actinobacteria^{34,35} as well as Proteinase K produced by the fungus Tritirachium album^{36,37}. Many of these proteases belong to the subtilase superfamily of serine proteases, e.g., Proteinase K³⁷ and subtilisins from Bacillus spp.³¹⁻³³. Despite the identification of several subtilases with activity against PLLA, the molecular mechanisms that are allowing these enzymes to have promiscuous activity against PLLA are currently poorly understood. Interestingly, Oda et al. (2000) had demonstrated PLLA depolymerizing capabilities by several commercially available subtilisins originating from Bacillus spp.; however, the activities were highly variable with some Bacillus subtilis showing little to no activity.

Bacillus species are major sources of enzymes with scientific and biotechnological applications due to their high growth rates yielding short fermentation times, their GRAS (generally regarded as safe) status with the FDA, and their ability to secrete enzymes directly into the extracellular medium^{38,39}. Furthermore, Bacillus species ability to secrete enzymes at grams per liter concentrations makes them ideal organisms for large scale enzyme production^{40,41}. Because of this, it is not surprising that a large portion of commercially available enzymes are produced either natively or heterologously by Bacillus species⁴². The infrastructure for the industrial production of Bacillus enzymes is well established.

EP1499717 describes a cytotoxicity assay that measures the release of a cytoplasmic component from dead and dying cells, wherein the preferred cytoplasmic component is lactate dehydrogenase (LDH). The methods set forth therein allow determining the cytotoxic effect of a test compound or set of test conditions.

### SUMMARY OF THE INVENTION

In an embodiment a modified subtilisin polypeptide comprising at least one amino acid substitution at a position selected from S33, T33, S62, Q62, S98, R98, T99, Y99, D101, S101, Q103, Y104, P129, N130, T130, E156, S156, T159, S159, T162, A187, S189, F189, T215, G215, Y217, N218, and T218 of a subtilisin polypeptide comprising an amino acid sequence selected from the group of subtilisin amino acid sequences set forth in SEQ ID NO:1 and SEQ ID NO:2 and combinations thereof is provided. In some aspects, the at least one amino acid substitution of the modified subtilisin polypeptide is selected from T33, Q62, R98, Y99, D101, Q103, Y104, P129, N130, S156, T159, T162, S189, T215, Y217, T218 of a subtilisin polypeptide comprising an amino acid sequence set forth in SEQ ID NO:1. The mature amino acid sequence of BpAprE is set forth in SEQ ID NO:1. In other aspects, the at least one amino acid substitution of the modified subtilisin polypeptide is selected from S33, S62, S98, T99, S101, T130, E156, S159, A187, F189, G215 and N218 of a subtilisin polypeptide comprising an amino acid sequence having the sequence set forth in SEQ ID NO:2. The mature amino acid sequence of BsAprE is set forth in SEQ ID NO:2.

In aspects of the modified subtilisin polypeptide, the amino acid substitution is selected from the group consisting of S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N. In certain aspects of the modified subtilisin polypeptide, the at least one amino acid substitution is selected from T33S, Q62S, R98S, Y99F, Y99W, Y99T, D101S, D101A, Q103S, Y104W, Y104F, P129A, P129S, N130T, N130S, S156A, S156E, T159F, T159S, T162F, S189F, T215G, T215A, Y217L, Y217F, T218S, and T218N of a subtilisin polypeptide comprising an amino acid sequence having the sequence set forth in SEQ ID NO:1. In other aspects of the modified subtilisin polypeptide, the at least one amino acid substitution is selected from S33T, S62Q, S98R, T99Y, S101D, T130N, E156S, S159T, A187N, F189S, G215T, and N218T of a subtilisin polypeptide comprising an amino acid sequence having the sequence set forth in SEQ ID NO:2.

In various aspects, the modified subtilisin polypeptide comprises at least two amino acid substitutions at a position selected from S33, T33, S62, Q62, S98, R98, T99, Y99, D101, S101, Q103, Y104, P129, N130, T130, E156, S156, T159, S159, T162, A187, S189, F189, T215, G215, Y217, N218, T218 of a subtilisin polypeptide comprising an amino acid sequence selected from the group of subtilisin amino acid sequences set forth in SEQ ID NO:1 and SEQ ID NO:2 and combinations thereof. In some aspects, the modified subtilisin polypeptide comprises at least three amino acid substitutions at a position selected from S33, T33, S62, Q62, S98, R98, T99, Y99, D101, S101, Q103, Y104, P129, N130, T130, E156, S156, T159, S159, T162, A187, S189, F189, T215, G215, Y217, N218, T218 of a subtilisin polypeptide comprising an amino acid sequence selected from the group of subtilisin amino acid sequences set forth in SEQ ID NO:1 and SEQ ID NO:2 and combinations thereof.

In certain aspects of the modified subtilisin polypeptides, the amino acid substitutions are selected from the group consisting of: S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N amino acid substitutions.

In various aspects, the modified subtilisin polypeptide comprises a combination of amino acid substitutions wherein the combination is selected from the group of combinations of amino acid substitutions consisting of:
(a) D101S, N130T and S189F;
(b) D101A, N130S and S189F;
(c) D101S, N130S and S189F;
(d) D101A, N130T and S189F;
(e) D101S, N130T, S189F and Y217L;
(f) S98R, S159T, N218T and A187N;
(g) S33T, T99Y and E156S;
(h) S159T and N218T;
(i) S98R and N218T;
(j) S33T, S98R, T99Y, E156S, S159T, and N218T;
(k) S33T, S98R, T99Y, E156S, S159T, A187N, and N218T;
(l) S33T and T99Y;
(m) S33T and E156S;
(n) S33T, T99Y and E156S;
(o) T99Y and E156S;
(p) D101S and N130T;
(q) D101A, N130T, S189F and Y217L;
(r) D101S, N130S, S189F and Y217L;
(s) D101S, N130T, S189F and Y217L;
(t) S98R and S159T;
(u) N130T and S189F; and combinations thereof.

In certain aspects, a modified subtilisin polypeptide of the application comprises a combination of amino acid substitutions selected from the group of combinations of amino acid substitutions consisting of:
(a) D101S, N130T and S189F;
(b) D101A, N130S and S189F;
(c) D1010S, N130S and S189F;
(d) D101A, N130T and S189F;
(e) D101S, N130T, S189F and Y217L;
(f) D101S and N130T;
(g) D101A, N130T, S189F and Y217L;
(h) D101S, N130S, S189F and Y217L;
(i) D101S, N130T, S189F and Y217L;
(j) N130T and S189F; and combinations thereof. Generally these combinations of mutations occur in a modified subtilisin polypeptide comprising an amino acid sequence set forth in SEQ ID NO:1.

In other aspects, a modified subtilisin polypeptide of the application comprises a combination of amino acid substitutions selected from the group of combinations of amino acid substitutions consisting of: (a) S98R, S159T, N218T and A187N;
(b) S33T, T99Y and E156S;
(c) S159T and N218T;
(d) S98R and N218T;
(e) S33T, S98R, T99Y, E156S, S159T, and N218T;
(f) S33T, S98R, T99Y, E156S, S159T, A187N, and N218T;
(g) S33T and T99Y;
(h) S33T and E156S;
(i) S33T, T99Y and E156S;
(j) T99Y and E156S;
(k) S98R and S159T;
(l) and combinations thereof. Generally these combinations of mutations occur in a modified subtilisin polypeptide comprising an amino acid sequence set forth in SEQ ID NO:2.

In various aspects, a modified subtilisin polypeptide comprising a combination of mutations further comprises at least one additional mutation selected from the group consisting of: S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N.

In some aspects, a modified subtilisin polypeptide of the application comprises a combination of amino acid substitutions selected from the group consisting of:
S98R, S159T, N218T and A187N;
S33T, S98R, T99Y, E156S, S159T, and N218T;
D101S, N130T, S189F and Y217L;
D101A, N130T, S189F and Y217L;
D101S, N130S, S189F and Y217L;
D101A, N130S, S189F and Y217L; and
S33T, S98R, T99Y, E156S, S159T, A187N, and N218T. In certain aspects, a modified subtilisin polypeptide comprising a combination of amino acid substitutions set forth above herein further comprises at least one additional mutation selected from the group consisting of: S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N.

In various aspects, any of the modified subtilisin polypeptides discussed above herein exhibit an altered poly(L) lactic acid (PLLA) depolymerization activity. In certain aspects the polypeptide exhibits an increased PLLA depolymerization activity. In some aspects, the increased PLLA depolymerization activity is at least 1.5× greater than the PLLA depolymerization activity of a subtilisin polypeptide having an amino acid sequence set forth in SEQ ID NO:1 or SEQ ID NO:2. In particular aspects, the increased PLLA depolymerization activity is at least 1.5× greater than the PLLA depolymerization activity of a subtilisin polypeptide having an amino acid sequence set forth in SEQ ID NO:1. In particular aspects, the increased PLLA depolymerization activity is at least 5× greater than the PLLA depolymerization activity of a subtilisin polypeptide having an amino acid sequence set forth in SEQ ID NO:2. In some aspects, the PLLA depolymerization activity is evaluated by a poly L lactic acid depolymerization assay. In certain aspects, the PLLA depolymerization activity is evaluated by quantification of L-lactate.

In various aspects the modified subtilisin polypeptide exhibits an altered protease activity. In some aspects, the modified subtilisin polypeptide exhibits an altered poly L lactic acid depolymerization activity and an altered protease activity.

In some aspects, the modified subtilisin polypeptide exhibits an increased PLLA depolymerization activity at least 1.5× greater than the PLLA depolymerization activity of a subtilisin polypeptide having an amino acid sequence set forth in SEQ ID NO:1 or SEQ ID NO:2, wherein the amino acid sequence comprises a combination of amino acid substitutions selected from the group of combinations consisting of (a) S33T, T99Y and E156S; (b) D101S and N130T and (c) D101S, N130T and S189F.

In various aspects, the modified subtilisin polypeptides exhibit an increased yield of soluble L lactate products. In some aspects, the modified subtilisin polypeptide exhibits a rapid depolymerization activity. In certain aspects, a modified subtilisin polypeptide exhibits increased depolymerization in less than 18 hours. In some aspects, the modified subtilisin polypeptide exhibits rapid depolymerization activity at a reaction temperature in the range of about 30° C. In some aspects, the modified subtilisin polypeptide exhibits a rapid depolymerization activity at a low reaction temperature.

In various aspects, the modified subtilisin polypeptide exhibits PLLA depolymerization activity at a pH between about 7 and about 10.5. In certain aspects, the modified subtilisin polypeptide exhibits increased PLLA depolymerization activity at approximately pH 9.5.

In various aspects, the modified subtilisin polypeptide exhibits increased PLLA depolymerization activity in the presence of 1 mM and 10 mM calcium salt. In certain aspects, the calcium salt is calcium chloride.

In an embodiment, methods of quantifying poly(L) lactic acid depolymerization are provided. Methods of quantifying poly(L) lactic acid depolymerization comprise the steps of (a) providing a plastic material comprising PLLA, (b) combining an enzyme of interest with the plastic material comprising PLLA; (c) incubating the plastic material comprising PLLA and enzyme of interest at a predetermined temperature; collecting the supernatant; inactivating the enzyme of interest; adding NAD+, L-lactate dehydrogenase, diaphorase and a reactive indicator to the supernatant; incubating a mixture comprising the supernatant, NAD+, L-lactate dehydrogenase, diaphorase and a reactive indicator; detecting the reactive indicator; determining the amount of L-lactate present; and quantifying the poly(L) lactic acid depolymerization from the amount of L-lactate present. In some aspects, the plastic material comprising PLLA is a film. In aspects of the methods, NAD+ and L-lactate dehydrogenase are added in excess. In various aspects of the methods, the reactive indicator is a redox-sensitive indicator, optionally resazurin. In some aspects, the step of detecting the reactive indicator comprises spectrophotometric detection. In certain aspects of the method, the method is quick, optionally wherein the method takes an hour or less. In other aspects of the method, incubating the PLLA and enzyme of interest occurs for approximately 15 to 17 hours at a predetermined temperature.

In some aspects of the methods, the method is highly sensitive. In certain aspects, the method has a minimum detection limit of about 1.5 μM L-lactate. In certain aspects, the method has a maximum detection limit near the saturating concentration of resorufin. In particular aspects, the method has a maximum detection limit of about 1.28 mM L-lactate.

In an embodiment, methods of depolymerizing PLLA are provided. Methods of depolymerizing PLLA comprise the steps of providing a plastic material comprising PLLA and incubating the plastic material with a modified subtilisin polypeptide of the application.

In an embodiment, methods of producing lactones from plastic comprising PLLA are provided. Methods of producing lactones from plastic comprising PLLA comprise the steps of (a) providing a plastic material comprising PLLA, (b) incubating the plastic material with a modified subtilisin polypeptide of the application and (c) collecting the lactones produced from the plastic material.

In an embodiment, an efficient method of recycling plastic material comprising PLLA is provided. The efficient method comprises the steps of (a) providing a plastic material comprising PLLA, (b) incubating the plastic material with a modified subtilisin of the application, (c) collecting the lactones produced from the material and (d) synthesizing the lactones into PLLA.

In an embodiment, methods of regenerating L-lactate monomers are provided. Methods of regenerating L-lactate monomers comprise the steps of (a) providing a plastic material comprising PLLA, (b) incubating the plastic material with a modified subtilisin of the application, and (c) collecting the l-lactate monomers regenerated from the plastic material.

Aspects of any of the methods may involve a step of incubating the plastic with a modified subtilisin that occurs at a predetermined temperature, optionally wherein the temperature is between about 25° C. and about 35° C. or wherein the temperature is about 30° C. In aspects of the methods, the plastic comprises at least 0.1% PLLA. In some aspects of the methods, the plastic comprises at least 1% PLLA. Various aspects of the methods further comprise the step of adjusting the pH to between about 7 and about 10.5. In certain aspects of the methods, incubating the plastic material with the modified subtilisin polypeptide occurs at a pH of about 9.5. Various aspects of the methods comprise adding calcium salt, optionally wherein the calcium salt is calcium chloride. In certain aspects of the methods, the calcium salt is between about 1 mM and about 10 mM.

In an embodiment, high-throughput methods of screening at least one enzyme for a PLLA depolymerizing activity are provided. The high-through put methods comprise performing a method of quantifying poly(L) lactic acid depolymerization in a multi-well microtiter plate. In an aspect of the high-throughput methods, the multi-well microtiter plate is selected from the group comprising a 96 well plate and a 96 well based plates.

### BRIEF DESCRIPTION OF DRAWINGS

FIGS. 1A and 1B present charts summarizing the PLLA depolymerization activity of wild-type subtilisins, BsAprE and BpAprE. FIG. 1A indicates the L-lactate (μM) observed in PLLA depolymerization reactions including two negative controls: a blank, Tris buffer and an empty vector control; and reaction mixtures containing either the wildtype BsAprE, wildtype BpAprE or proteinase K enzyme preparations. FIG. 1B indicates the L-lactate (μM)/μg protease observed in PLLA depolymerization reactions with the indicated crude enzyme extract.

FIGS. 2A, 2B, 2C, 2D and 2E provide tables summarizing various activities of modified BsAprE subtilisin polypeptides. The altered amino acid residues in each modified BsAprE subtilisin polypeptide variant are indicated on the x-axis. FIG. 2A provides the L-lactate μM/μg protease for site directed amino acid variants having an alteration selected from the following: S33T, S62Q, T99Y, S101D, T130N, E156S and F189S. Results obtained from wildtype BsAprE and BpAprE are shown in FIG. 2A. Results that are not significantly different from WT BsAprE are indicated with “ns”; results that are significantly different are indicated with “****”. FIG. 2B presents the L-lactate μM/μg protease for site directed amino acid variants having an alteration selected from the following: S33T, S62Q, T99Y, S101D, T130N, and F189S without including the results from E156S or the BpAprE wild-type subtilisin. When the E156S or the BpAprE wild-type subtilisin data are removed, the statistically significant increase in the S33T variant is apparent. FIG. 2C presents the L-lactate μM/μg protease results for the E156S engineered polypeptide and the BpAprE wild-type subtilisin. The E156S engineered polypeptide exhibits statistically significant increased activity as compared to the BpAprE wild-type subtilisin. FIG. 2D summarizes protease results obtained from the indicated variants tested with N-succinyl-(Ala)₃-pNA as the substrate. The percent activity standardized to the wild-type average is shown on the y-axis. The Bs-based subtilisin is indicated on the x-axis. Significant results are indicated with “****”. FIG. 2E summarizes protease results obtained from the indicated variants tested with N-succinyl-AAPF-pNA as the substrate. The percent activity standardized to the wild-type average is shown on the y-axis. The Bs-based subtilisin is indicated on the x-axis. Significant results are indicated with “****”.

FIGS. 3A and 3B summarize various activities of modified BpAprE subtilisin polypeptides. The altered amino acid residues in each modified BpAprE subtilisin polypeptide variant are indicated on the x-axis. The L-lactate μM/μg protease values are shown on the y-axis of each chart. FIG. 3A includes results from the D101S and S189F variants. FIG. 3B focuses on results from the T33S, Q62S, Y99T, N130T and S156E variants. Results from wild-type BpAprE and BsAprE are shown in both FIGS. 3A and 3B.

FIG. 4 provides ribbon diagrams and models of the BsAprE, BsAprE S156S, BpAprE polypeptides. The model views are shown from the front and side, with an expanded image of the binding pocket region showing the distance from the amino acid at residue 156 to the proline at residue 129.

FIG. 5 provides ribbon diagrams and models of the BpAprE, BpAprE D101S and BsAprE polypeptides. The model views are shown from the front and side, with an expanded image of the binding pocket region showing the distance from the amino acid at residue 101 to the proline at residue 129.

FIGS. 6A, 6B, 6C and 6D summarize results obtained from combinations of individual mutations such as double and triple mutations. A synergistic effect is observed with at least the BsAprE T99Y/E156S and S33T/T99Y/E156S variants (FIGS. 6A and 6B) The S33T/T99Y/E156S variant showed an 830.3 fold change relative to wildtype; this change is significantly greater than the sum of the change seen with the individual mutations (FIG. 6B) or even the double S33T/T99Y added to the change seen in the E156S variant. The L-lactate μM/μg protease values are shown on the y-axis of FIGS. 6A and 6C. The fold change relative to wildtype of the variant's is presented in FIGS. 6B and 6D. A synergistic effect is observed with at least the BpAprE D101S/N130T, N130T/S189F and D101S/N130T/S189F variants (FIGS. 6C and 6D).

FIG. 7 provides images of PLLA films after the indicated treatment. Blank Tris buffer is the negative control and proteinase K is the positive control. Visible depolymerization (cloudiness) is present in PLLA films treated with a BpAprE D101S/N130T/S189F engineered subtilisin.

FIG. 8 presents a schematic of the L-lactate reaction mechanism and conversion of the reactive indicator, resazurin, to resorufin and an image of the L-lactate standard curve.

FIG. 9 provides an image of a gel. The gel was loaded with approximately 10 μg protein from TCA precipitated supernatants from bacterial cultures transformed with either an empty expression vector or an expression vector encoding a subtilisin homolog.

FIG. 10 summarizes the percent of activity against PLLA standardized to wildtype BpAprE activity (y-axis) of the indicated variant (x-axis). Results from the BpAprE triple mutant, N130S, D101A, S156A, P129A, P129S, Y104W, Y104F, Q103S, Y99F, Y99W, T218S, T162F, T159F, T215G, T215A, Y217L and Y217F variants are presented. Assays were run for 2 hours in 100 mM Tris pH 9.0 buffer. Many individual mutations significantly reduced the activity below the level exhibited by the wild-type subtilisin. The Y217L variant showed substantially increased activity while Y217F showed a significant decrease in activity.

FIGS. 11A and 11B summarizes results obtained with the D101S/N130T/S189F BpAprE triple variant and a quadruple D101S/N130T/S189F/Y217L variant after incubating reactions for two hours (FIG. 11A) or overnight (FIG. 11B).

FIGS. 12A-12C summarize results from experiments to optimize the BpAprE reaction evaluating pH levels (FIG. 12A), ethanolamine buffer concentration (FIG. 12B) and Ca²⁺ concentration (FIG. 12C). The assays were run for 2 hours at 30° C. The boxes indicate original parameters. The percent activity against PLLA normalized to the original parameter is shown on the y-axis.

FIG. 13 presents results of LC-MS analysis of the D101S/N130T/S189F BpAprE triple mutant L-lactate product sizes.

FIGS. 14A and 14B present results obtained from PLLA depolymerization assays with the indicated variant. FIG. 14A summarizes results obtained from BsAprE variants including the BsAprE triple variant, S98R, S159T, G215T and N218T. FIG. 14B summarizes results obtained from BpAprE variants including the BpAprE triple variant, R98S, T159S, T215G and T218N.

FIGS. 15A and 15B present results obtained from PLLA depolymerization assays with the indicated variant. S98R, S159T, N218T, and A187N were introduced into the BsAprE Triple variant individually and in the indicated combinations (x-axis). The BsAprE Triple plus S98R, S159T, N218T variant (BsAprE sextuple) exhibited PLLA depolymerization activity (y-axis, L-lactate μM/μg protease), near that of the BpAprE Triple (FIGS. 15A and 15B). The BsAprE sextuple A187N variant exhibited activity not significantly different from the BpAprE Triple. The PLLA depolymerization activity of the BsAprE sextuple, BsAprE sextuple A187N variant and BpAprE Triple exceeded the PLLA depolymerization activity of proteinase K.

### DETAILED DESCRIPTION OF THE INVENTION

Recycling via enzymatic depolymerization has recently emerged as a promising as this can regenerate L-lactate monomers that can be re-used in PLLA synthesis²³. Also, this process can be performed under mild reaction conditions with lower energy input and fewer harmful chemicals relative to chemical and mechanical practices for PLLA recycling^{21,23,24}. Identification and characterizing of novel PLLA depolymerizing enzymes may provide a diverse array of tools that may be useful in enzymatic recycling processes. However, the promiscuous nature of naturally-occurring PLLA depolymerizing enzymes, whose primary functions are as proteases^{25,26} and esterases^{26,27}, lends to low catalytic efficiencies against non-naturally occurring PLLA polymers²⁸. It thus becomes essential to engineer these enzymes to have increased catalytic capabilities against their intended polymer to improve efficiencies of these processes. Prior to Applicants' work, there were no studies demonstrating protein engineering efforts for PLLA depolymerizing enzymes. Elucidating the molecular mechanisms affecting polymer depolymerizing activity may aid in rationale design studies for enzyme engineering. Understanding the mechanisms allowing for some subtilisins to depolymerize PLLA, but not other subtilisins, provides novel insights into substrate preference in subtilisins and bolsters rationale design approaches for engineering subtilisins for increased activity against PLLA.

Applicants have identified a subtilisin from a soil isolate of Bacillus pumilus B12⁴³, hereby denoted as BpAprE, that has the ability to degrade high molecular weight PLLA. To better understand the structural details of BpAprE that are allowing it to depolymerize PLLA, we performed a comparative analysis with a homolog of this enzyme, AprE from Bacillus subtilis PY79 (BsAprE) that has limited ability to degrade PLLA despite showing strong protease activity. We generated mutations in the active sites of both enzymes to make them more similar to each in an attempt to identify the residues that are favored for PLLA depolymerization. In doing so, we have unexpectedly determined that both enzymes have residues favored for the process leading to the generation of variants with greatly enhanced activity relative to the wildtype versions of the enzymes. Without being limited by mechanism, in silico protein modeling suggests that opening of the binding pocket and increases in surface hydrophobicity may explain the increases in activity in these enzymes. Finally, combinations of mutations that increase activity appear to have synergistic effects, leading to the visible depolymerization of PLLA by at least one of the novel BpAprE variants described herein.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term about is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

Ranges may be expressed herein as from “about” one particular value, and/or to “about” another particular value. When such a range is expressed, +/−5% or more preferably +/−2% is included. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Any embodiment of any of the disclosed methods or compositions can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

By “comprising” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. polyadenylation sequence, enhancer elements, other genes, linker domains, etc.

By “consisting essentially of”, it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g. linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of”, it is meant the exclusion from the composition, method, or kit of any element, step, or ingredient not specified in the claim. For example, an expression cassette “consisting of” a gene encoding a therapeutic polypeptide operably linked to a promoter, and a polyadenylation sequence consists only of the promoter, polynucleotide sequence encoding the therapeutic polypeptide, and polyadenlyation sequence. As another example, a polypeptide “consisting of” a recited sequence contains only the recited sequence.

The feature or features of one embodiment may be applied to other embodiments, even though not described or illustrated, unless expressly prohibited by this disclosure or the nature of the embodiments.

### Definitions

Amino acid: As used herein, the term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” or “non-natural amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide. It should be noted that all amino acid residue sequences are represented herein by formulae whose left and right orientation is in the conventional direction of amino-terminus to carboxy-terminus.

Expression cassette: An “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

Modified subtilisin: As used herein, the term “modified subtilisin” refers to a subtilisin originated from another (i.e., parental) subtilisin and contains one or more amino acid alterations (e.g., amino acid substitution, deletion, or insertion) compared to the parental subtilisin. In some embodiments, a modified subtilisin of the invention is originated or modified from a naturally-occurring or wild-type subtilisin. In some embodiments, a modified subtilisin of the invention is originated or modified from a recombinant or engineered subtilisin including, but not limited to, chimeric subtilisin, fusion subtilisin or another modified subtilisin.

Mutation: As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence or an alteration of a function, property, phenotype or trait found in the protein encoded by the parental sequence. It is recognized that an alteration in one phenotype may be an increase or a decrease and that alterations to a second phenotype may or may not occur and may or may not occur in the same direction as the alteration of the first phenotype.

Nucleic Acid Molecule: The term “nucleic acid molecule” includes RNA, DNA and cDNA molecules. It will be understood that, as a result of the degeneracy of the genetic code, a multitude of nucleotide sequences encoding a given protein such as alpha-hemolysin and/or variants thereof may be produced. The present invention contemplates every possible variant nucleotide sequence, encoding variant alpha-hemolysin, all of which are possible given the degeneracy of the genetic code.

Promoter: As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

Purified: As used herein, “purified” means that a molecule is present in a sample at a concentration of at least 95% by weight, or at least 98% by weight of the sample in which it is contained.

Purifying: As used herein, the term “purifying” generally refers to subjecting transgenic nucleic acid or protein containing cells to biochemical purification and/or column chromatography.

Variant: The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a polypeptide variant comprises at least one amino acid difference (e.g., amino acid substitution, amino acid insertion, amino acid deletion) relative to a reference polypeptide sequence, e.g. a native polypeptide sequence, and a polynucleotide variant comprises at least one nucleotide or nucleoside difference (e.g., nucleotide or nucleoside substitution, insertion, or deletion) relative to a reference polynucleotide sequence, e.g., a native polynucleotide sequence.

Variant subtilisin: The term “variant subtilisin gene” or “variant subtilisin” means, respectively, that the nucleic acid sequence of a subtilisin gene from Bacillus has been altered by removing, adding, and/or manipulating the coding sequence or the amino acid sequence of the expressed protein has been modified consistent with the invention described herein.

Vector: As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

Wild-type: As used herein, the term “wild-type” refers to a gene or gene product which has the characteristics of that gene or gene product when isolated from a naturally-occurring source.

Percent homology: The term “% homology” is used interchangeably herein with the term “% identity” herein and refers to the level of nucleic acid or amino acid sequence identity between the nucleic acid sequence that encodes any one of the inventive polypeptides or the inventive polypeptide's amino acid sequence, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive polypeptides, as described herein.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.)

A preferred alignment of selected sequences in order to determine “% identity” between two or more sequences, is performed using for example, the CLUSTAL-W program in MacVector version 13.0.7, operated with default parameters, including an open gap penalty of 10.0, an extended gap penalty of 0.1, and a BLOSUM 30 similarity matrix.

### Nomenclature

In the present description and claims, the conventional one-letter and three letter codes for amino acid residues are used.

For ease of reference, variants of the application are described by use of the following nomenclature: Original amino acid(s); position(s); substituted amino acid(s). According to this nomenclature, for instance, the substitution of a valine by a lysine in position 149 is shown as: Val149Lys or V149K. Multiple mutations are separated by/signs, such as: His35Gly/Val149Lys or H35G/V149K representing mutations in positions 35 and 149 substituting glycine for histidine and lysine for valine, respectively.

As used herein, the term “gene product” refers to the desired expression product of a polynucleotide sequence such as a peptide or protein.

As used herein, the terms “polypeptide” and “protein” refer to polymers of amino acids of any length. The term “peptide” refers to a polymer of amino acids of about 50 or fewer amino acids. The terms also encompass an amino acid polymer that has been modified, as by for example, disulfide bond formation, glycosylation, lipidation, or phosphorylation. In some instances, a subject polypeptide may have a length of greater than 50 amino acids.

As used herein, the term “operably linked” refers to a juxtaposition of genetic elements, e.g. promoter, enhancer, termination signal sequence, polyadenylation sequence, etc., wherein the elements are in a relationship permitting them to operate in the expected manner. For instance, a promoter is operably linked to a coding region if the promoter helps initiate transcription of the coding sequence. There may be intervening residues between the promoter and coding region so long as this functional relationship is maintained.

PLLA depolymerization assay: As used herein, a “PLLA depolymerization assay” refers to any means of assessing the depolymerization of poly(L)-Lactic acid (PLLA) in the presence of an enzyme. Examples of suitable reaction conditions for a PLLA depolymerization assay are provided elsewhere herein.

Tools used in the investigation of polymer depolymerization include both qualitative, measures such as, but not limited to, scanning electron microscopy and clearing of polymer emulsions and quantitative measures. Quantitative measures for testing polymer depolymerization often test bulk changes in the polymer while not explicitly testing products from the depolymerization reaction, e.g., atomic force microscopy and Fourier transform infrared spectroscopy. Many studies have demonstrated the effectiveness of testing reaction products of PLLA depolymerization, i.e., L-lactate, through LC-MS^{32,53} or commercially available L-lactate assay kits^{36,43}. However, these methodologies greatly limit high throughput processes partly due to associated costs. Additionally, previous L-lactate assays described in the literature for use in PLLA depolymerization can be labor intensive^{53,54} reducing the throughput of these methodologies.

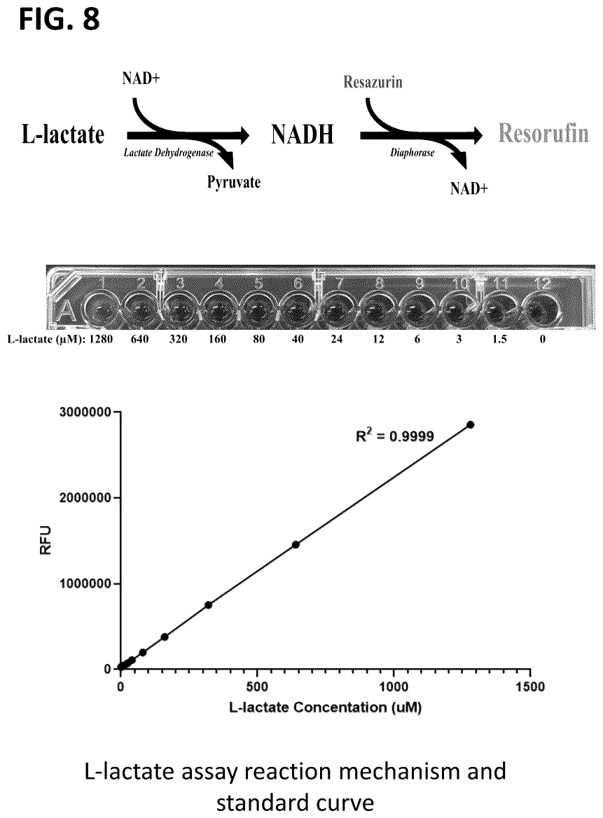

A foundational aspect of this work was the development of a sensitive, rapid, and cost-effective enzymatic L-lactate assay for the purposes of quantifying the end-product of PLLA depolymerization, i.e., L-lactate (FIG. 8). The new enzymatic assay consists of a single reaction mixture in which lactate dehydrogenase converts L-lactate in crude samples to pyruvate with the coupled reduction of NAD+ to NADH. Diaphorase then re-oxidizes NADH to NAD+ with the concomitant reduction of resazurin. The fluorogenic end product, resorufin, is detected spectrophotometrically (FIG. 8). We have optimized the reagents and reaction conditions relative to those described in Shapiro and Silanikove (2010)⁵⁵ to better suit our purposes while simultaneously minimizing cost. Specifically, the reactants NAD+ and L-lactate dehydrogenase are added in excess to drive the formation of pyruvate while simultaneously minimizing the amount of diaphorase to reduce costs associated with the assay. The resulting assay is a quick (1 hour), highly sensitive (R2=0.9999) assay with a detection limit as low as 1.5 μM and as high as 1.28 mM before reaching a saturating concentration of resorufin end product (FIG. 8). One limitation of this assay is that bacterial strains may ferment sugars to L-lactate creating confounding results with L-lactate detection. When using crude enzyme preparations, a washout step may be utilized, or alternatively the L-lactate in no PLLA controls may be utilized. As far as we know, this is the first time an L-lactate assay has been described for use in the investigation of PLLA depolymerization. The currently described assay can be run in a 96-well plate format for high throughput screening of PLLA depolymerizing variants.

PLLA films are known in the art. Methods of producing PLLA films are known in the art. Solidified PLLA film for use in PLLA depolymerization assays was prepared by dissolving PLLA and allowing the PLLA to resolidify into a PLLA film.

Reactive indicators include but are not limited to redox sensitive indicator such as resazurin. The end product of resazurin reduction is resorufin, a spectrophotometrically detectable substance.

Multi-well microtiter plates are known in the art and are commercially available from a variety of sources. Multi-well microtiter plates include, but are not limited to, 48 well plates, 96 well plates and 96-well based plates including but not limited to 192 well plates, 288 well plates, 384 well plates, 480 well plates, 672 well plates, 768 well plates, and 1536 well plates.

### Site-Directed Mutagenesis of Subtilisin

The B. pumilus AprE and B. subtilis AprE genes wild-type amino acid sequences are provided here (SEQ ID NO:3 and SEQ ID NO:4, respectively) and elsewhere. The wild-type polypeptides are processed into mature proteins having the mature amino acid sequences set forth in SEQ ID NO:1 and SEQ ID NO:2, respectively. Methods of making point mutations are known in the art. Any method of introducing point mutations known in the may be used in making variants of either the BpAprE or BsAprE gene. Methods of making variants may include, but are not limited to, PCR based methods, primer extension PCR, inverse PCR mutagenesis, and overlap extension PCR, QuikChange and SPRINP. Kits for performing various types of site directed mutagenesis are commercially available.

The modified subtilisin proteins provided herein include specific substitutions or one or more combinations of substitutions. The modified subtilisin proteins were evaluated for PLLA depolymerization activity. Some of the modified subtilisin proteins were evaluated for protease activity against other substrates as well.

In certain embodiments, the modified subtilisin protein includes one or more mutations at one or more of the locations of an amino acid sequence set forth in either SEQ ID NO:1 or SEQ ID NO:2. The term “modified subtilisin protein” may be used interchangeably with any of the following terms: “subtilisin variant”, “modified subtilisin polypeptide”, “modified subtilisin”, “variant subtilisin”, “variant subtilisin polypeptide”, “engineered subtilisin”, “engineered variant subtilisin”, “engineered subtilisin variant”, “engineered subtilisin polypeptide” and “engineered subtilisin protein”. In certain example embodiments, the modified subtilisin formed from mutating one or more of the amino acids of SEQ ID NO:1 or SEQ ID NO:2 has 80%, 85%, 90%, 91%, 92%, 93%, 94%, 95%, 96%, 97%, 98%, 99% or more identity to a subtilisin sequence set forth in either SEQ ID NO:1 or SEQ ID NO:2. Amino acid substitutions may include at least one amino acid substitution at a position selected from S33, T33, S62, Q62, R98, S98, T99, Y99, D101, S101, Q103, Y104, P129, N130, T130, E156, S156, T159, S159, T162, A187, S189, T215, G215, Y217, N218, and T218. In some aspects, the at least one amino acid substitution is selected from T33, Q62, R98, Y99, D101, Q103, Y104, P129, N130, S156, T159, T162, S189, T215, Y217, T218 of a subtilisin polypeptide comprising an amino acid sequence set forth in SEQ ID NO:1. In other aspects, the at least one amino acid substitution is selected from S33, S62, S98, T99, S101, T130, E156, S159, A187, F189, G215 and N218 of a subtilisin polypeptide comprising an amino acid sequence having the sequence set forth in SEQ ID NO:2.

In certain example embodiments, the variant may include a particular amino acid substitution. For example the variant may include an amino acid substitution of any one of S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N or combinations thereof. In various aspects, the amino acid substitution is selected from T33S, Q62S, R98S, Y99F, Y99W, Y99T, D101S, D101A, Q103S, Y104W, Y104F, P129A, P129S, N130T, N130S, S156A, S156E, T159F, T159S, T162F, S189F, T215G, T215A, Y217L, Y217F, T218S, and T218N of a subtilisin polypeptide comprising an amino acid sequence having the sequence set forth in SEQ ID NO:1 In certain aspects, the amino acid substitution is selected from the group consisting of S33T, S62Q, S98R, T99Y, S101D, T130N, E156S, S159T, A187N, F189S, G215T, and N218T of a subtilisin polypeptide comprising an amino acid sequence having the sequence set forth in SEQ ID NO:2.

Modified subtilisins may be a single variant, double variant, triple variant, quadruple variant, sextuple variant, septuple variant, octuple variant. Modified subtilisins may comprise a single variant, double variant, triple variant, quadruple variant, sextuple variant, septuple variant and an octuple variant. Modified subtilisins may further comprise one or more additional mutations or combinations of mutations.

Combinations of mutations of interest, include but are not limited to the following combinations: (a) D101S, N130T and S189F;
(b) D101A, N130S and S189F;
(c) D101S, N130S and S189F;
(d) D101A, N130T and S189F;
(e) D101S, N130T, S189F and Y217L;
(f) S98R, S159T, N218T and A187N;
(g) S33T, T99Y and E156S;
(h) S159T and N218T;
(i) S98R and N218T;
(j) S33T, S98R, T99Y, E156S, S159T, and N218T;
(k) S33T, S98R, T99Y, E156S, S159T, A187N, and N218T;
(l) S33T and T99Y;
(m) S33T and E156S;
(n) S33T, T99Y and E156S;
(o) T99Y and E156S;
(p) D101S and N130T;
(q) D101A, N130T, S189F and Y217L;
(r) D101S, N130S, S189F and Y217L;
(s) D101S, N130T, S189F and Y217L;
(t) S98R and S159T;
(u) N130T and S189F; and combinations thereof. Any of the above combinations may be further combined with one or more additional site-directed mutations described herein, including any of 533, T33, 562, Q62, R98, S98, T99, Y99, D101, 5101, Q103, Y104, P129, N130, T130, E156, 5156, T159, 5159, T162, A187, 5189, T215, G215, Y217, N218, and T218; particularly any one of S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N. It is recognized that certain combinations of mutations demonstrate higher depolymerization activity than variants with other combinations of mutations.

Double variants of particular interest include, but are not limited to S159T/N218T, S98R/N218T, S33T/T99Y, S33T/E156S, D101S/N130T, N130T/S189F, T99Y/E156S, and S98R/S159T. It is recognized that D101S/N130T, N130T/S189F may occur in the context of a subtilisin comprising the amino acid sequence set forth in SEQ ID NO:1. It is recognized that S159T/N218T, S98R/N218T, S33T/T99Y, S33T/E156S, T99Y/E156S and S98R/S159T may occur in the context of a subtilisin comprising the amino acid sequence set forth in SEQ ID NO:2.

Triple variants of particular interest include, but are not limited to, D101S/N130T/S189F; D101A/N130S/S189F; D101S/N130S/S189F; D101A/N130T/S189F; and S33T/T99Y/E156S. It is recognized that a modified subtilisin polypeptide comprising an amino acid sequence set forth in SEQ ID NO:1 may include a triple mutation selected from the triple mutation combinations: D101S/N130T/S189F; D101A/N130S/S189F; D101S/N130S/S189F and D101A/N130T/S189F. It is recognized that a modified subtilisin polypeptide comprising an amino acid sequence set forth in SEQ ID NO:2 may include a triple mutation such as but not limited to the S33T/T99Y/E156S combination.

Quadruple variants of particular interest include, but are not limited to, D101S/N130T/S189F/Y217L; S98R/S159T/N218T/A187N; D101A/N130T/S189F/Y217L; D101S/N130S/S189F/Y217L; and D101S/N130T/S189F/Y217L. It is recognized that a modified subtilisin polypeptide comprising an amino acid sequence set forth in SEQ ID NO:1 may include a quadruple mutation selected from the quadruple mutation combinations: D101S/N130T/S189F/Y217L; D101A/N130T/S189F/Y217L; D101S/N130S/S189F/Y217L; and D101S/N130T/S189F/Y217L. It is recognized that a modified subtilisin polypeptide comprising an amino acid sequence set forth in SEQ ID NO:2 may include a quadruple mutation such as but not limited to the S98R/S159T/N218T/A187N combination.

Sextuple variants of particular interest include, but are not limited to, S33T/S98R/T99Y/E156S/S159T/N218T. It is recognized that a modified subtilisin polypeptide comprising an amino acid sequence set forth in SEQ ID NO:2 may include a sextuple mutation such as but not limited to the S33T/S98R/T99Y/E156S/S159T/N218T combination. Septuple variants of particular interest include, but are not limited to, S33T/S98R/T99Y/E156S/S159T/A187N/N218T. It is recognized that a modified subtilisin polypeptide comprising an amino acid sequence set forth in SEQ ID NO:2 may include a septuple mutation.

It is further recognized that any double, triple, quadruple, sextuple or septuple combination may be further combined with an additional single mutation and/or any double, triple, quadruple, sextuple or septuple combination described herein.

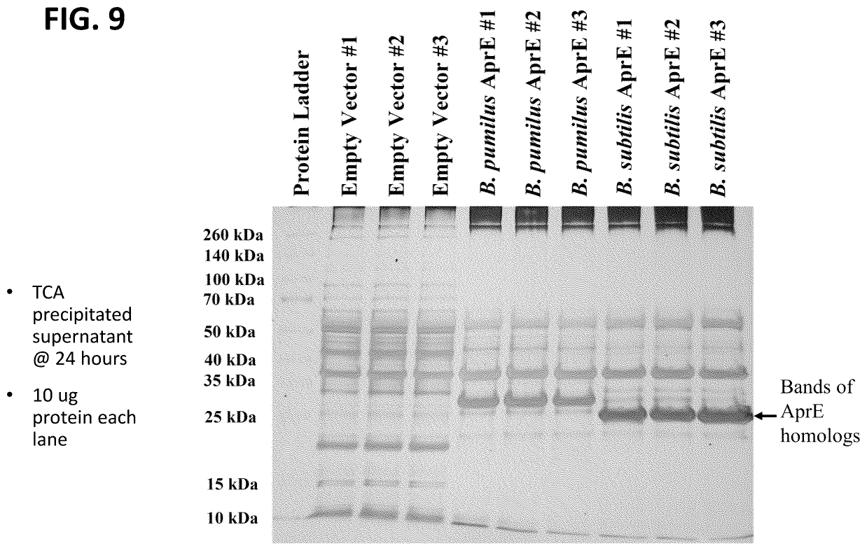

Cloning and screening of the B. subtilis AprE and B. pumilus AprE genes was carried out in the protease-deficient strain B. subtilis WB800 to easily screen for functional, secreted proteases without any background protease activity. Importantly, SDS-PAGE and silver stain analysis demonstrate a distinct band forming for these two proteins at their expected molecular weight (FIG. 9), thus validating the standardization method based on band intensity described in the examples.

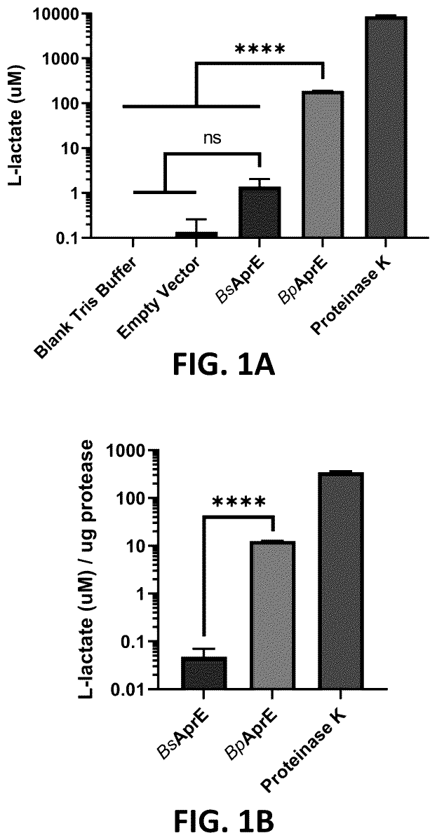

Incubation of crude, concentrated enzyme preparations containing either BpAprE or BsAprE subtilisin with PLLA shows that BpAprE has a statistically significant, higher amount of L-lactate produced relative to BsAprE, empty vector, and blank buffer controls (p-value=<0.0001) (FIG. 1A). Notably, BsAprE shows no statistically significance relative to the empty vector control or blank buffer despite having a greater average value. When standardized to protease concentration, BpAprE has activity that is greater than 2-orders of magnitude higher than BsAprE (p value=<0.0001) (FIG. 1B). Wild-type BpAprE has the ability to depolymerize high molecular weight PLLA while wild-type BsAprE has little to no ability to depolymerize PLLA. When compared to Proteinase K, BpAprE shows ^{~}40-fold less activity against PLLA, indicating that it is a less potent enzyme for PLLA depolymerization. Interestingly, despite this drastic difference in PLLA depolymerization activity, BsAprE and BpAprE share 76% amino acid sequence identity. When considering residues with similar biochemical properties (as indicated by alignment in Clustal Omega), these enzymes share 92% sequence similarity. These similarities are further underpinned by both enzymes sharing nearly identical abundances of acid, basic, hydrophobic, and neutral residues.

An alignment between the two enzymes in their processed form without their secretion signals or autoprocessed propeptide domains was created⁴⁰. Sequences of 582 BpAprE homologs sharing ≥50% sequence identity were aligned to determine conserved residues among the enzymes. Homology modeling identified a putative active site of the enzyme. Interestingly, only 7 residues within the putative active sites of these enzymes were different, and many of these different residues shared similar biochemical characteristics, e.g., polar, hydroxyl containing residues at position 33 for both enzymes. Only 12 of 28 residues are conserved in the active site based on the alignment, with the other 16 residues showing only a limited number of residues that occur at any given position (^{~}2-4 residues). BsAprE and BpAprE exhibit vastly different PLLA depolymerizing capabilities even though the two enzymes are closely related structurally.

By “altered poly(L) lactic acid (PLLA) depolymerization activity” is intended an alteration in an enzyme's ability to degrade high molecular weight PLLA. The alteration may be an increase in PLLA depolymerization activity, a decrease in PLLA depolymerization activity, a change in the reaction kinetics, a change in the I-lactone product size distribution (such as an altered distribution of monomers and dimers), or any other change to the enzyme's ability to degrade high molecular weight PLLA. The alteration is evaluated as compared to the activity of a subtilisin having the wild-type mature amino acid sequence. Depolymerization of high molecular weight PLLA yields low molecular weight PLLA such as soluble L-lactate products. Soluble L-lactate products may include, but are not limited to monomers, dimers, trimers, small multimers (such as 4, 5, 6, 7, 8, 9 or 10 mers) and mixtures thereof.

The polypeptides having the wild-type mature amino acid sequences set forth in SEQ ID NO:1 or SEQ ID NO:2 exhibit different PLLA depolymerization activities. It is understood that modified polypeptides having amino acid modifications are compared to the modified polypeptides original wild-type mature amino acid sequence. For example, a modified subtilisin polypeptide having one or more amino acid substitutions in a subtilisin polypeptide having an amino acid sequence set forth in SEQ ID NO:1 may exhibit an altered PLLA depolymerization activity as compared to a subtilisin polypeptide having the amino acid sequence set forth in SEQ ID NO:1, but the modified subtilisin polypeptide having one or more amino acid substitutions in a subtilisin polypeptide having an amino acid sequence set forth in SEQ ID NO:1 may not exhibit an altered PLLA depolymerization activity as compared to a subtilisin polypeptide having the amino acid sequence set forth in SEQ ID NO:2. Similarly a modified subtilisin polypeptide having one or more amino acid substitutions in a subtilisin polypeptide having an amino acid sequence set forth in SEQ ID NO:2 may exhibit an altered PLLA depolymerization activity as compared to a subtilisin polypeptide having the amino acid sequence set forth in SEQ ID NO:2, but the modified subtilisin polypeptide having one or more amino acid substitutions in a subtilisin polypeptide having an amino acid sequence set forth in SEQ ID NO:2 may not exhibit an altered PLLA depolymerization activity as compared to a subtilisin polypeptide having the amino acid sequence set forth in SEQ ID NO:1. Alternatively, a modified subtilisin polypeptide having one or more amino acid substitutions in a subtilisin polypeptide having an amino acid sequence set forth in either SEQ ID NO:1 or SEQ ID NO:2, may exhibit an altered PLLA depolymerization activity as compared to a subtilisin polypeptide having the amino acid sequence set forth in SEQ ID NO:2 and a subtilisin polypeptide having the amino acid sequence set forth in SEQ ID NO:1. A modified subtilisin polypeptide having one or more amino acid substitutions in a subtilisin polypeptide having an amino acid sequence set forth in either SEQ ID NO:1 or SEQ ID NO:2, may not exhibit an altered PLLA depolymerization activity as compared to either a subtilisin polypeptide having the amino acid sequence set forth in SEQ ID NO:2 or a subtilisin polypeptide having the amino acid sequence set forth in SEQ ID NO:1.

The activity of a modified subtilisin polypeptide is always compared to the activity of a subtilisin polypeptide having the parental amino acid sequence. A modified subtilisin polypeptide that exhibits an altered PLLA depolymerization activity may have an increased PLLA depolymerization activity. An increased PLLA depolymerization activity may be increased at least about 1.2×, 1.3×, 1.4× 1.5×, 1.6×, 1.7×, 1.8×, 1.9×, 2×, 2.5×, 3×, 3.5×, 4×, 4.5×, 5×, 5.1×, 5.5×, 6×, 6.5×, 7×, 7.5×, 8×, 8.5×, 9×, 9.5×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 72×, 74×, 76×, 78×, 80×, 82×, 84×, 86×, 88×, 90×, 92×, 94×, 96×, 98×, 100×, 105×, 110×, 115×, 120×, 125×, 130×, 135×, 140×, 145×, 150×, 155×, 160×, 165×, 170×, 175×, 180×, 185×, 190×, 195×, 200×, 205×, 210×, 215×, 220×, 225×, 230×, 235×, 240×, 245×, 250×, 255×, 260×, 265×, 270×, 275×, 280×, 285×, 290×, 295×, 300×, 350×, 400×, 450×, 500×, 550×, 600×, 650×, 700×, 750×, 800×, 830×, 850×, 900×, to at least 1000× as compared to the activity of the wild-type mature polypeptide.

A modified subtilisin polypeptide of the application may exhibit an altered protease activity. The altered protease activity may be an altered activity against a substrate such as, but not limited to, a subtilisin substrate, a polypeptide comprising L-alanine residues, N-succinyl-AAPF-pNA and N-succinyl-(Ala)₃-pNA.

The PLLA depolymerization activity is evaluated by a poly(L) Lactic acid (PLLA) depolymerization assay as described elsewhere herein (See FIG. 8). In some aspects, the PLLA depolymerization is evaluated by quantification of L-lactate. In some aspects, a PLLA depolymerization assay comprises a step of quantifying L-lactate. Methods of quantifying L-lactate are known in the art; a lactate quantification kit is available from BioAssays.

A modified subtilisin polypeptide may exhibit an increased yield of soluble L Lactate products. A modified subtilisin polypeptide may exhibit an increased yield of l-lactate monomers.

A modified subtilisin polypeptide may exhibit rapid depolymerization activity. By rapid depolymerization activity is intended an increased activity against PLLA in less than five hours, less than 4 hours, less than 3 hours, less than 2 hours or less than 1 hour after combining the modified subtilisin polypeptide with PLLA. A rapid depolymerization activity reaches at least 1%, 5%, 10%, 20%, 25%, 30%, 35%, 40%, 45%, or 50% completion sooner than the wild-type subtilisin. A rapid depolymerization activity may reach at least 0.1% completion within about 1 hour, 2 hours, 3 hours, 4 hours or about 5 hours. A rapid depolymerization activity may be at least 1%, 5%, 10%, 20%, 25%, 30%, 35%, 40%, 45%, or 50% higher than a wild-type subtilisin within about 1 hour, 2 hours, 3 hours, 4 hours or about 5 hours.

A modified subtilisin polypeptide may exhibit increase depolymerization after an incubation of less than about 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, and 1 hour.

A modified subtilisin polypeptide may exhibit rapid depolymerization at a low reaction temperature. A modified subtilisin polypeptide may exhibit increased depolymerization at a low reaction temperature. By low reaction temperature is intended a temperature between about 10° C. and about 50° C., between about 10° C. and 45° C., between about 15° C. and about 40° C., between 20° C. and about 35° C., between about 25° C. and about 35° C., or about 30° C.

A modified subtilisin polypeptide may exhibit PLLA depolymerization activity between about pH 7 and pH 10.5, between about pH 8 and pH10, between about pH 8.5 and about 10, and between about pH 9 and about pH 10. A modified subtilisin polypeptide may exhibit increased PLLA depolymerization activity between about pH 7 and pH 10.5, between about pH 8 and pH10, between about pH 8.5 and about 10, and between about pH 9 and about pH 10. A modified subtilisin polypeptide may exhibit increased PLLA depolymerization activity at about pH 9.5.

A modified subtilisin polypeptide may exhibit PLLA depolymerization activity in presence of between about 1 mM and 100 mM calcium salt, between about 1 mM and about 50 mM calcium salt, between about 1 mM and about 30 mM calcium salt, between about 1 mM and about 20 mM calcium salt, and between about 1 mM and about 10 mM calcium salt. A modified subtilisin polypeptide may exhibit increased PLLA depolymerization activity between about 1 mM and about 30 mM calcium salt, between about 1 mM and about 20 mM calcium salt, and between about 1 mM and about 10 mM calcium salt. A modified subtilisin polypeptide may exhibit increased PLLA depolymerization activity at about 1 mM, 2 mM, 3 mM, 4 mM, 5 mM, 6 mM, 7 mM, 8 mM, 9 mM or about 10 mM calcium salt. Calcium salts are known in the art and include, but are not limited to, calcium chloride.

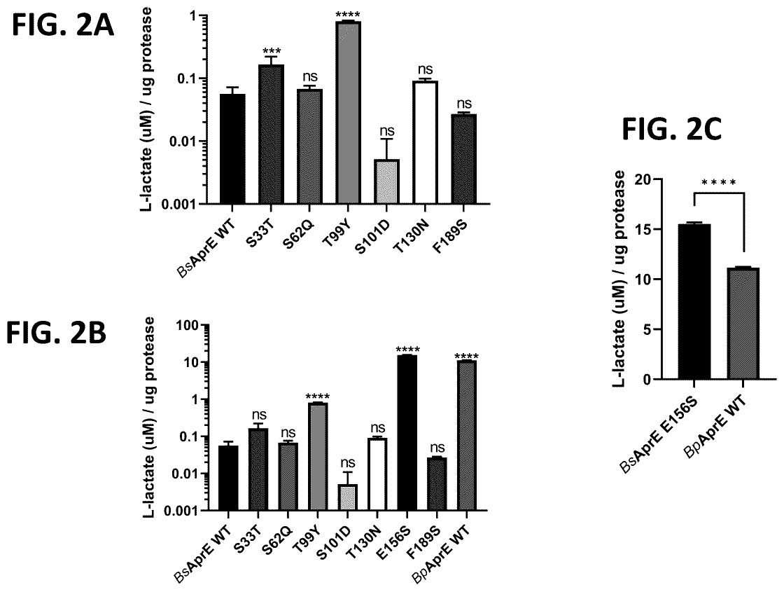

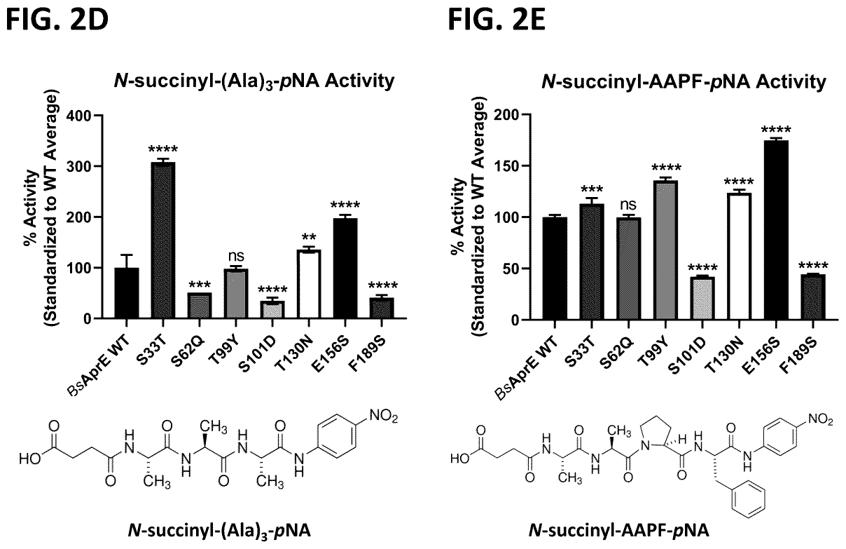

Single amino acid substitutions were made in BsAprE to make it more similar to BpAprE. Activity of the mutants against PLLA showed several residues with statistically significant increases in activity relative to BsAprE wildtype enzyme, namely the mutations T99Y and E156S (FIG. 2A). S33T did not appear statistically significance activity relative to wildtype due to the large values of E156S and BpAprE; however, removal of these groups showed that the S33T mutation was statistically increased relative to wildtype BsAprE (FIG. 2B). Strikingly, the E156S mutation of BsAprE increased over 2 orders of magnitude and surpassed the activity of the BpAprE (FIG. 2C). Without being limited by mechanism, the glutamate at the 156 position may be a critical limiting factor with regard to the ability of a subtilisin to depolymerize PLLA. Glutamate 156 is the preferred amino acid at this position followed by serine and alanine. The E156S mutation relative to BpAprE suggests that BsAprE has residues favored for PLLA depolymerization that are not present in BpAprE. The S101D and F189S variants exhibit a lower average standardized L-lactate production that may indicate favored residues in wild-type BsAprE.

Variant enzymes were tested against two chromogenic protease substrates, N-succinyl-AAPF-pNA and N-succinyl-(Ala)₃-pNA. N-succinyl-AAPF-pNA is a subtilisin substrate and many of its amino acids have hydrophobic characteristics. N-succinyl-(Ala)₃-pNA is a known substrate to several PLLA depolymerizing proteases. Without being limited by mechanism, PLLA depolymerization may occur due to substrate promiscuity toward L-alanine type residues—an amino acid with structural similarities to L-lactate^{64,65}. Variant enzymes with changes in activity against PLLA were expected to show similar trends to changes in activity to N-succinyl-(Ala)3-pNA; unexpectedly, this was not the case (FIG. 2D). The E156S variant exhibited a 2-fold increase in activity, but there was no significant increase for T99Y relative to BsAprE wildtype (FIG. 2E). Surprisingly, while only showing a slight increase in activity for PLLA (FIG. 2A-2B), BsAprE S33T showed a substantial, 3-fold increase in activity against the substrate N-succinyl-(Ala)3-pNA (FIG. 2E). These results suggest that S33T has an increased preference for L-alanine-based substrate, though this increase only weakly translates to a preference for L-lactate-based substrate. These results suggests N-succinyl-(Ala)3-pNA is a weak indicator of PLLA depolymerization activity, unlike previous work⁶⁴. Serine to threonine may increase pocket hydrophobicity with the addition of the methyl group of threonine, while still maintaining any hydrogen bonding potential with the hydroxyl group. Interestingly, N-succinyl-AAPF-pNA actually trends better with PLLA depolymerization (FIG. 2E), suggesting that a large hydrophobic amino acid at the P1 position of the substrate may be preferred.

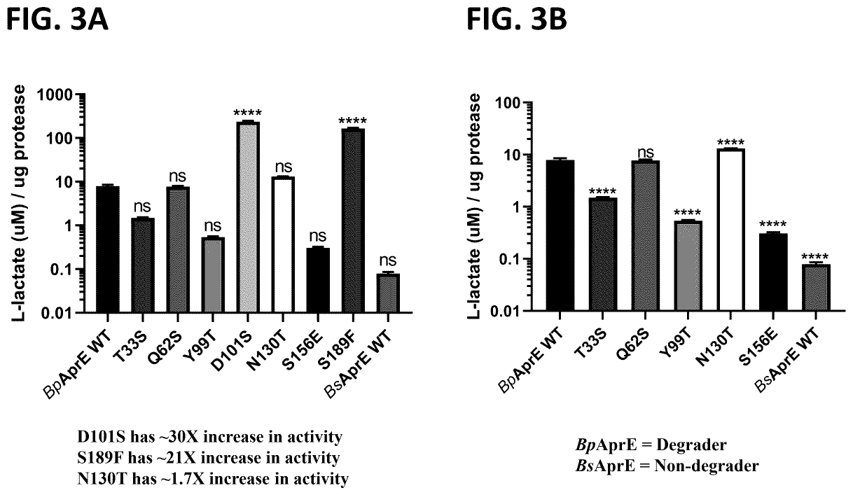

To further assess whether residues within BsAprE are actually favored for PLLA depolymerization, we generated single amino acid mutations in BpAprE to make it more similar to BsAprE (FIG. 3A-3B). There were substantial increases in activity for D101S (^{~}30-fold) and S189F (^{~}21-fold) mutations, with a modest increase in activity for N130T (^{~}1.7-fold) (FIG. 3A). These results demonstrate that enzymes with low activity may still have residues favored for the process. Interestingly, some BsAprE and BpAprE mutations showed a reciprocal relationship to one another in which mutations that increased activity in one caused a decrease in activity in the other, and vice versa (FIG. 2A-2E and FIG. 3A-3B). These changes in activity also appeared to occur to similar extents. For example, BsAprE saw an increase in activity in S33T, T99Y, and E156S (from smallest increase to largest), and BpAprE saw a decrease in activity in these residues following a similar trend of T33S, Y99T, and E156S (from smallest decrease to largest). Another striking similarity between these two data sets is that when residues were changed to threonine in BsAprE S33T and BpAprE N130T, small increases in activity were observed suggesting threonine may be a residue favored for PLLA depolymerization activity in some circumstances. The next largest increase in activity in both data sets, BsAprE T99Y and BpAprE S189F, showed that introducing large, hydrophobic amino acids to the protein can substantially increase activity. Introducing large hydrophobic amino acids to the protein is a common modification used in the engineering of plastic depolymerizing enzymes. Unexpectedly, the largest activity increases were observed with BsAprE E156S and BpAprE D101S. BsAprE E156S and BpAprE D101S are both mutations that change large, negatively charged amino acids and turn them into serine—a small, polar amino acid.

In silico protein modeling reveals structural changes associated with increased enzymatic activity.

Mutations that showed an increase in activity in BsAprE and BpAprE were introduced into in silico enzyme models to determine if any structural changes were associated with increases in activity. BsAprE E156S, the variant with a striking increase in activity, shows a slight opening of one of the enzymes binding pockets. Using MOE, we measured the distance across the binding pocket using the two closest atoms. In this model, we can see that the BsAprE E156S mutation increases the distance across the binding pocket from 3.77 Å to 4.43 Å and is visibly more open in our model. However, this increase in distance is not substantial enough to meet that of BpAprE which shows a distance of 5.72 Å across the binding pocket. Without being limited by mechanism, opening of the binding pocket may makes the hydrophobic residues within (surface labeled in green) more accessible, potentially increasing binding to the hydrophobic PLLA surface. This hypothesis is further supported by the increase in activity against the substrate N-Succinyl-AAPF-pNA by BsAprE E156S relative to BsAprE wildtype (FIG. 2E). Residue number 156 is part of the S1 binding pocket of subtilisins⁵⁸ and associates with the amino acid at the P1 position on protease substrate, i.e., phenylalanine on N-Succinyl-AAPF-pNA. An increase in activity against this substrate would suggest an opening of this binding pocket and unveiling of hydrophobic residues to accommodate the large, hydrophobic phenylalanine residue.

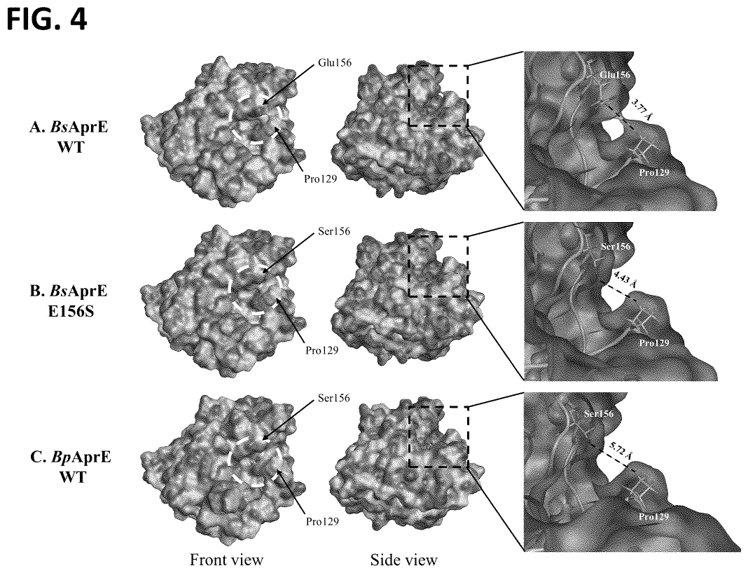

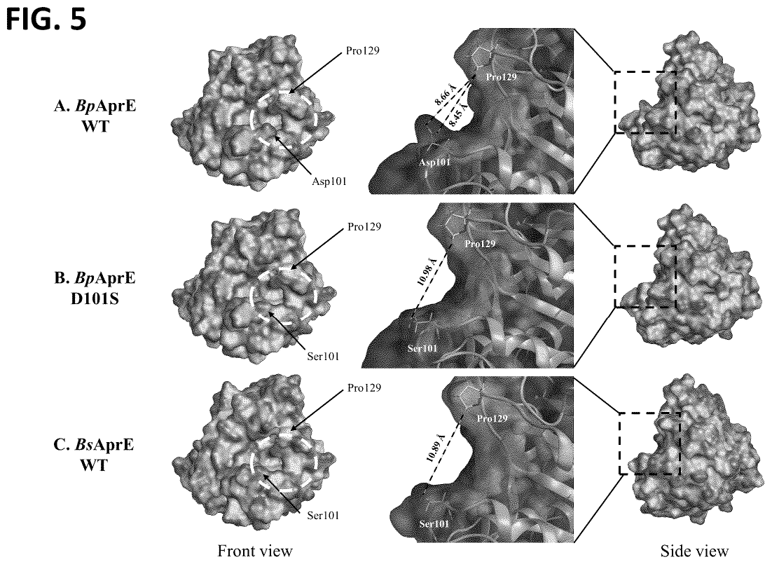

Similarly, BpAprE D101S, the mutant with the greatest increase in activity among the BpAprE mutants, may further support the idea that an opening of the binding pocket increases PLLA depolymerization activity. Measuring the distance between both oxygens of the carboxylate group in Asp101 in BpAprE wildtype, as well as the hydroxyl group of Ser101 in the BpAprE D101S mutant, with the closest hydrogen atom of Pro129 across the binding pocket revealed an increase in binding pocket width associated with the D101S substitution (FIG. 5). The distance across the pocket increases from 8.66 Å/8.45 Å for Asp101 carboxylate group to 10.98 Å in the Ser101 group, becoming remarkably similar to the BsAprE wildtype which has a distance 10.89 Å between Ser101 and Pro129 (FIG. 5). The position of Asp101 in BpAprE wildtype appears to be oriented in a way that may sterically hinder the binding of the enzyme active site to the PLLA surface; however, the substitution with serine at this position appears to mitigate this hinderance (FIG. 5). Another striking similarity between BsAprE E156S and BpAprE D101S is that Pro129 is the closest residue across the binding pocket for both enzymes (FIGS. 4 and 5). As this is a non-conserved residue in subtilisins, modification of this residue to something smaller and less rigid than proline, such as alanine or serine as seen in other subtilisins, may lead to further increases in activity due to further broadening of this pocket. PLLA depolymerization may be favored in subtilisins with more open binding pockets, potentially allowing for better binding to the substrate.

Modifying enzyme surface hydrophobicity to increase interaction between the substrate and enzyme surface is a strategy that has been utilized for engineering of plastic degrading enzymes²⁸. BsAprE T99Y may be a mutation that follows a similar mechanism as this tyrosine residue is positioned just outside of the binding pocket on the front face of BsAprE. Without being limited by mechanism, the residue in this position could potentially increase binding with the surface of PLLA, allowing enhanced contact between the active site and the polymer chain. Similarly BpAprE S189F, in which the planar, hydrophobic Phe189 is positioned just outside of the binding pocket on the front face of the enzyme, may potentially alter substrate binding. These explanations are supported by the associated increase in activity against PLLA seen in both of these mutations (FIGS. 2 and 3).

BsAprE S33T produced a visually undetectable change in the active site topology of the enzyme and was thus not further pursued based on structural information. Structural details pertaining to BpAprE N130T are discussed elsewhere herein.

### Multiple Mutations Act Synergistically to Increase PLLA Depolymerization Activity

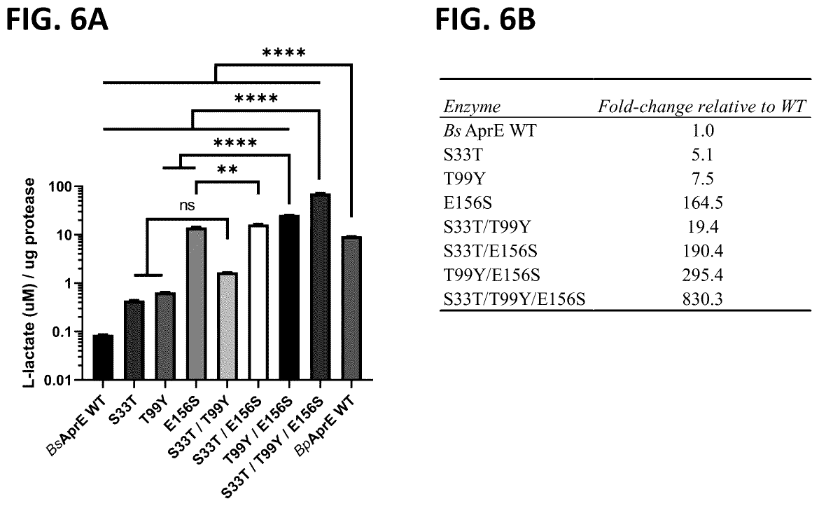

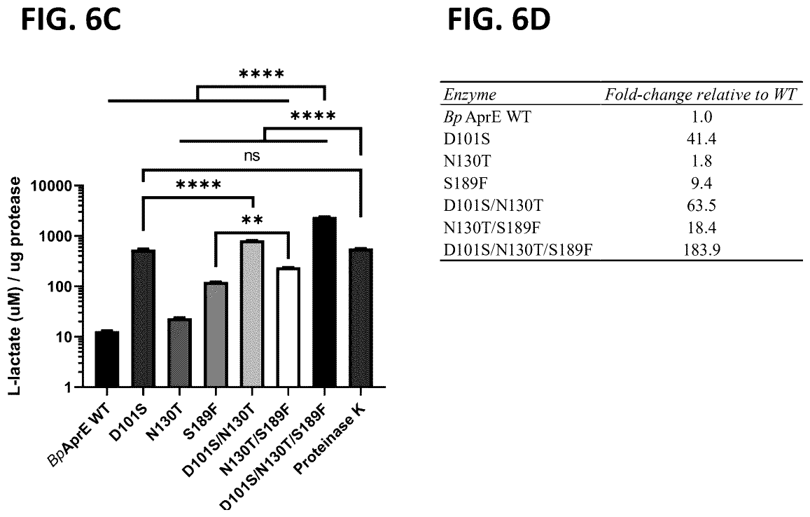

Double and triple mutants for both BsAprE and BpAprE (FIGS. 6A-6D) were made. With the exception of BsAprE S33T/T99Y, all double mutants and the triple mutant showed a statistically significant increase in activity relative to their constituent single mutations alone. The insignificance in BsAprE S33T/T99Y relative to its constituent single mutations may be due to the large values associated with the double and triple mutants containing E156S as a higher fold change relative to wildtype in the S33T/T99Y double mutant relative to S33T and T99Y alone is exhibited (FIG. 6B). Notably, all E156S containing mutations show a statistically higher activity against PLLA than BpAprE (p-value=<0.0001) (FIG. 6A). Furthermore, it appears that combining mutations has a synergistic effect, as opposed to additive, in which the fold-change in activity is substantially higher than the combined fold-changes associated with each constituent mutation (FIG. 6B). This point is most clearly shown in BsAprE S33T/T99Y/E156S in which there is an 830-fold increase relative to wildtype BsAprE while an additive effect of constituent mutations would produce an enzyme with less than 200-fold increase in activity (FIG. 6B). BsAprE S33T/T99Y/E156S produces ^{~}71 μM/ug protease which greatly surpasses BpAprE WT which shows ^{~}7.5-fold less activity (FIG. 6A). The synergistic effects seen in BsAprE S33T/T99Y/E156S are remarkable.

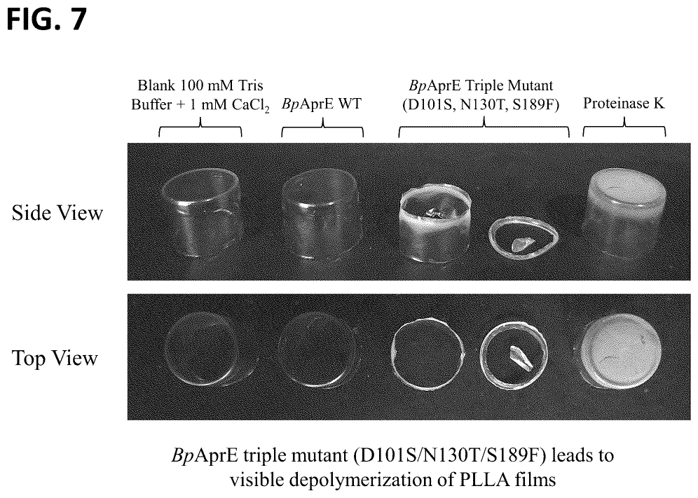

Combining mutations in BpAprE showed a similar synergistic effect in which combined mutations show a significantly greater amount of activity relative to constituent mutations alone (FIG. 6C-6D). Furthermore, the fold changes in the BpAprE double and triple mutants were higher than what would be expected from adding the constituent mutations together (FIG. 6D). An example of this is seen in BpAprE N130T which only shows a modest increase in activity of 1.8-fold relative to BpAprE wildtype, but increases BpAprE D101S/N130T to 63.5-fold higher than wildtype relative to 41.4-fold in BpAprE D101S (FIG. 6D). Strikingly, BpAprE D101S has a 41.4-fold increase in activity against PLLA relative to wildtype yet shows no significant difference when compared to proteinase K (FIG. 6C). Combining other mutations with BpAprE D101S significantly increases its activity relative to its constituent mutations and significantly surpasses the activity of Proteinase K (FIG. 6C). The synergistic effects of BpAprE D101S/N130T/S189F lead to a 183.9-fold increase in activity that is 4.2-fold higher than that of proteinase K (FIGS. 6C-6D). The significant increase in activity of BpAprE D101S/N130T/S189F relative to BpAprE wildtype and Proteinase K is exemplified by substantial visual depolymerization of high molecular weight PLLA films when incubated with crude protein extracts containing comparable amounts of protease (FIG. 7). Relative to a blank tris buffer negative control, the BpAprE wildtype enzyme showed virtually no signs of depolymerization as the film remained completely intact and transparent (FIG. 7). BpAprE D101S/N130T/S189F and proteinase K both showed clear signs of depolymerization, with an opaqueness forming on both samples that without being limited by mechanism, may be the result of holes and pits forming in the plastic leading to a diffraction of light (FIG. 7). BpAprE D101S/N130T/S189F further appears to have a solubilization effect in which much of the bottom surface of the PLLA film was turned into soluble L-lactate products (FIG. 7). The visibly higher amount of depolymerization occurring at the bottom of the polymer is likely a result of static incubations with crude enzyme preparations. Notably, this substantial amount of depolymerization occurred over a short time scale (overnight ^{~}16 hours) and was done at the relatively low temperature of 30° C.

Without being limited by mechanism, one possible explanation for the synergistic effects seen when combining mutations is that these mutations may work together to enhance favorable structural features. Combining mutations N130T and D101S in BpAprE, led to a greater opening of the binding pocket than with the either mutation alone. Distances were measured from the hydroxyl and carboxyl groups of serine and aspartate at the 101 position, respectively, to both functional groups on the terminal ends of the side chains of asparagine and threonine in the 130 position. BpAprE N130T led to a slight increase in the distance between side chains across the pocket relative to BpAprE wildtype from the 8.91 Å/10.13 Å to 9.26/11.54 Å, respectively. BpAprE D101S showed an even greater increase in distance relative to BpAprE wildtype becoming 12.16 Å/13.66 Å, which is in line with our previous model of BpAprE D101S (FIG. 5). When combining BpAprE N130T and D101S, the distances show a further increase relative to BpAprE D101S alone becoming 13.10 Å/15.37 Å, becoming comparable to the BsAprE wildtype distances of 14.15 Å/15.26 Å (FIG. 9). The increase in distance across the binding pocket with the combined mutations may explain why BpAprE N130T, which only has a 1.8-fold increase in activity relative to BpAprE wildtype, can have a substantial effect when combined with other mutations (FIGS. 6C-6D). Furthermore, the conversion of residues with bulky side chains to smaller residues produces a favorable effect when they are associated with the edges of binding pockets (FIGS. 4 and 5).

Mutational synergism that enhances activity against PLLA occurs within the subtilisin family and may extend to other enzymes that depolymerize PLLA and/or enzymes that depolymerize other plastics. The addition of more favorable mutations into both BsAprE and BpAprE has the potential to create an even more potent variant against PLLA, even if the mutation alone has a small effect as seen in BpAprE N130T. We used a comparative analysis between BsAprE and BpAprE in this study to elucidate structural and mechanistic details favored for PLLA depolymerization. Notably, altering 6 of 7 non-conserved residues showed significant changes in activity relative to wildtype. Mutation of the remaining 8 non-conserved residues may identify favorable mutations for PLLA depolymerization. Amino acid variants that expand binding pocket width and/or introduce hydrophobic amino acids within and around the binding pocket may improve activity. Aside from active site residues, it is clear that there are likely non-active site residues that are different between BsAprE and BpAprE that are leading to vastly different activities. The active sites of BsAprE S33T/T99Y/E156S and BpAprE D101S/N130T/S189F only differ by single amino acid at the 62 position, which did not show any differences in activity when mutated in either enzyme (FIGS. 2 and 3). However, BsAprE S33T/T99Y/E156S only produced ^{~}70 μM per μg protease and BpAprE D101S/N130T/S189F produced ^{~}2.4 mM per μg protease suggesting that non-active site residues between the enzymes are contributing to this large difference in activity.

Modified Bacillus pumilus AprE Subtilisin Variants

Provided herein are mutations made to Bacillus pumilus subtilisin (BpAprE) that lead to a ^{~}184-fold increase and activity and the visible depolymerization of poly-L-lactic acid (PLLA). These mutations include D101S/N130T/S189F, and this engineered enzyme is also referred to as “BpAprE Triple.”

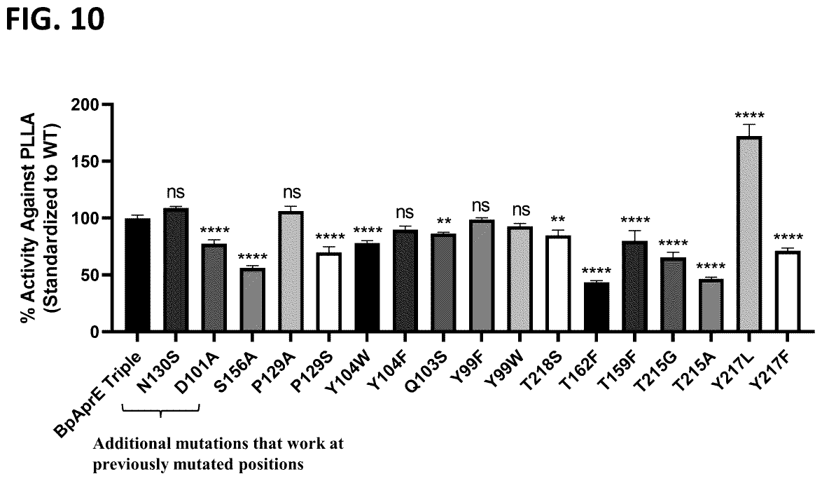

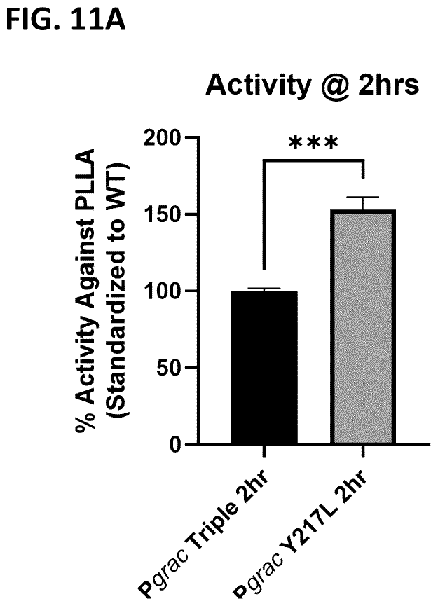

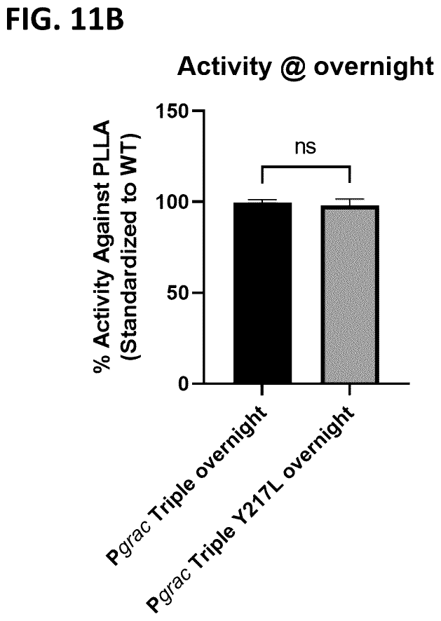

Also included herein are 17 mutations to the BpAprE Triple enzyme to further engineer the protein for increased activity (FIG. 10). An additional mutation at the 130 amino acid position (N130S) maintains the increase in activity seen in the D101S/N130T/S189F BpAprE Triple variant. Similarly, a mutation at the 101 amino acid position (D101A) maintains approximately 80% of the activity of the D101S/N130T/S189F BpAprE Triple variant and is still a substantial increase from wildtype BpAprE without any other mutations. Another subtilisin variant with increased activity is the Y217L/D101S/N130T/S189F modified subtilisin protein. The Y217L/D101S/N130T/S189F modified subtilisin protein exhibits a significant change in activity (approximately 60% increase) relative to the D101S/N130T/S189F modified subtilisin protein when standardized to enzyme concentration. The Y217L/D101S/N130T/S189F variant's increased activity occurred mostly in the short term (2 hours) and did not have an increase in the long term (overnight) (FIG. 11).

Modified Bacillus subtilis AprE Subtilisin Variants

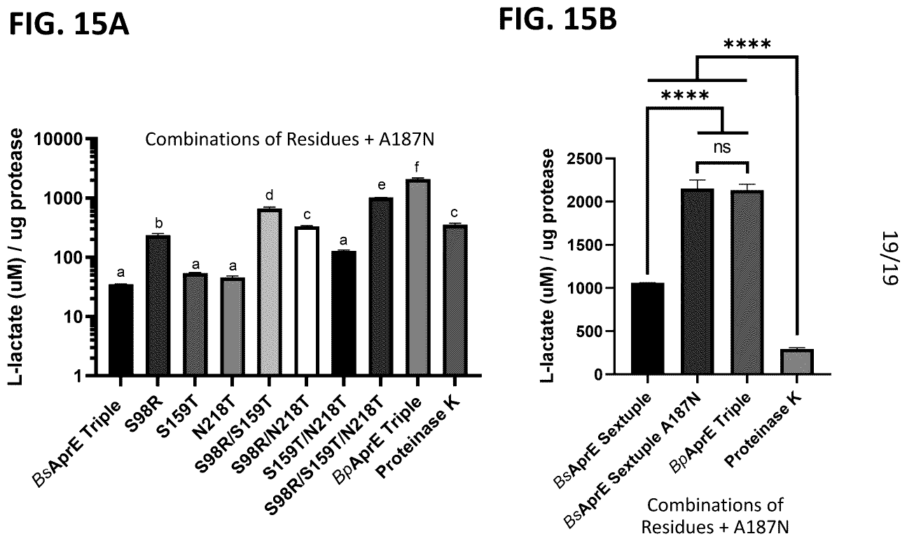

Additional mutations to the Bacillus subtilis subtilisin (BsAprE) show substantial increases in activity and bring the enzyme closer in activity to the potent BpAprE Triple variant (FIG. 5). These mutations were added to Bacillus subtilis subtilisin that contained mutations S33T/T99Y/E156S, which increased activity by ^{~}830-fold. The S33T/T99Y/E156S variant is also known as BsAprE Triple. Mutations added to BsAprE Triple, S98R, S159T, N218T, and A187N showed increases in activity relative to BsAprE Triple. Mutations made to BsAprE Triple that showed increases in activity were combined to engineer a more potent variant of the enzyme, and the variants were evaluated. All mutations combined with S98R lead to significant increases in activity relative to their respective single mutation alone. Only the combination of S159T/N218T did not show a statistically significant increase in activity relative to single constituent mutations alone (FIG. 15A). However, S159T/N218T had a ^{~}3.7× increase in activity relative to ^{~}1.6× and ^{~}1.3× increase for S159T and N218T, respectively, suggesting these combinations may still have biologically significant despite lack of statistical significance. Notably, BsAprE Triple S98R/N218T matches Proteinase K in terms of activity and is not significantly different in our analysis (FIG. 15A). BsAprE Triple S98R/S159T/N218T yielded a ^{~}30-fold increase in activity relative to BsAprE Triple and is ^{~}50% as potent as BpAprE with ^{~}2.9× more activity than Proteinase K based on L-lactate release (FIG. 15A). BsAprE Triple S98R/S159T/N218T only demonstrated about half the activity of BpAprE Triple suggesting the presence of additional amino acid(s) that are favored for activity in BpAprE that BsAprE does not have. We identified residue 187 as a potential target and introduced the mutation A187N to BsAprE in combination with all previously made mutations (S33T/T99Y/E156S/T159S/S98R/N218T) and found that this increased the activity by 50% and matches the activity of BpAprE Triple (FIG. 15B).

Methods of quantifying poly(L) lactic acid depolymerization are provided herein. The PLLA depolymerization assay comprises the steps of providing a plastic material comprising PLLA, combining an enzyme of interest with the plastic material comprising PLLA, incubating the plastic material comprising PLLA and enzyme of interest a predetermined temperature, collecting the supernatant, inactivating the enzyme of interest, adding NAD+, L-lactate dehydrogenase, diaphorase and a reactive indicator to the supernatant; incubating the mixture comprising the supernatant, NAD+, L-lactate dehydrogenase, diaphorase and a reactive indicator; detecting the reactive indicator and quantifying the poly(L) lactic acid depolymerization from the reactive indicator. Any enzyme of interest may be evaluated for PLLA depolymerization activity using the claimed method. Enzymes of particular interest include the subtilisins and modified subtilisin polypeptides as described elsewhere herein.

Methods of quantifying poly(L) lactic acid depolymerization are provided herein. The PLLA depolymerization assay comprises the steps of providing a PLLA film, combining an enzyme of interest with the PLLA film, incubating the PLLA film and enzyme of interest for a predetermined temperature, collecting the supernatant, inactivating the enzyme of interest, adding NAD+, L-lactate dehydrogenase, diaphorase and a reactive indicator to the supernatant; incubating the mixture comprising the supernatant, NAD+, L-lactate dehydrogenase, diaphorase and a reactive indicator; detecting the reactive indicator and quantifying the poly(L) lactic acid depolymerization from the reactive indicator. Any enzyme of interest may be evaluated for PLLA depolymerization activity using the claimed method. Enzymes of particular interest include the subtilisins and modified subtilisin polypeptides as described elsewhere herein.

Methods of inactivating an enzyme of interest are known in the art. Any method of deactivating an enzyme of interest suitable for use in the assay may be utilized.

“Diaphorase”: By “diaphorase” is intended any enzyme able to oxidize a reduced form of the coenzyme NAD. Diaphorase may also be referred to as cytochrome b5 reductase, NADH dehydrogenase, NADPH dehydrogenase, lipoyl dehydrogenase or dihydrolipoamide dehydrogenase. Diaphorases are known in the art and commercially available. Diaphorase may be added in the range of about 0.01 U/ml to about 2 U/ml, in the range of about 0.02 U/ml to about 1.5 U/ml, in the range of about 0.03 U/ml to about 1 U/ml, in the range of about 0.04 U/ml to about 0.5 U/ml, in the range of about 0.04 to about 0.1 U/ml, in the range of about 0.04 to about 0.07 U/ml, or in the range of about 0.05 U/ml. In some aspects of the methods, diaphorase is added at low levels compared to previous L-lactate assays. See EP1499717 and the commercially available product Bioassay Systems EnzyFluo™ L-Lactate Assay Kit. Surprisingly the L-lactate assay of the current application is highly sensitive and able to detect from about 1.5 μM to about 1280 μM L-Lactate.

NAD+: By “NAD+” is intended the oxidized form of nicotinamide adenine dinucleotide, a coenzyme. NAD+ may be added to reactions “in excess”, meaning that the amount of NAD+ added to the reaction is greater than could be used by the other reactants. Similarly in some aspects of the methods, more L-lactate dehydrogenase is added to the reaction than could be used by other reactants. Neither NAD+ nor L-lactate dehydrogenase are limiting factors in the reaction. The balanced lactate dehydrogenase reaction would equate to approximately a 1:1 ratio of NAD+ to Lactate, the same concentration of NAD+ and lactate would be balanced. Any amount of NAD+ above the 1.28 mM lactate detection limit may be considered “in excess”. Concentrations of NAD+ that are “in excess” may be in the range of at least 2 mM, at least 3 mM, at least 4 mM, at least 5 mM, at least 6 mM, at least 7 mM, at least 8 mM, at least 9 mM, at least 10 mM or greater. Concentrations of L-lactate dehydrogenase that are “in excess” may be in the range of about 0.05 U/ml, 0.1 U/ml, 1 U/ml, 2 U/ml, 3 U/ml, 4 U/ml, 5 U/ml, 6 U/ml, 7 U/ml, 8 U/reaction, 9 U/reaction, 10 U/reaction, or greater. Concentrations of L-lactate dehydrogenase “in excess” are in comparison to the diaphorase concentration.

In various aspects of the methods, the step of detecting the reactive indicator comprises spectrophotometric detection. Methods of spectrophotometric detection are known in the art. Any method of spectrophotometric detection capable of detecting in the appropriate wavelength may be utilized in the methods of the application.

Various aspects of the methods include methods that are highly sensitive. Highly sensitive methods are capable of detecting very low levels of PLLA depolymerization reactions including but not limited to a minimum detection limit of at least about 1.0 μM, 1.5 μM, 2.0 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM or more. In some instances highly sensitive methods are capable of detecting high levels of PLLA depolymerization including, but not limited to a maximum detection limit near the saturation concentration of the reactive indicator. In those methods where the reactive indicator is resorufin, the maximum detection limit may be up to about 1.3 mM, 1.28 mM, 1.26 mM, 1.24 mM, 1.2 mM, 1.0 mM, 0.8 mM, 0.6 mM, 0.4 mM, 0.2 mM, 0.1 mM or less.

Plastic materials comprising PLLA are known in the art. Plastic materials comprising PLLA may often be formed by 3D printing type techniques. PLLA forms a highly regular stereocomplex with increased crystallinity. PLLA can be used in plastics for applications ranging from use as medical implants to piezo-electric applications. The temperature stability of plastics comprising PLLA can be increased by mixing PLLA with PDLA at ratios up to 1:1, but much lower levels of PDLA also improve stability of plastic materials comprising PLLA. Plastic materials comprising at least about 0.01% PLLA, at least about 0.1% PLLA, at least about 1% PLLA, at least about 2% PLLA, at least about 3% PLLA, at least about 4% PLLA, at least about 5% PLLA, at least about 6% PLLA, at least about 7% PLLA, at least about 8% PLLA, at least about 9% PLLA, at least about 10% PLLA, at least about 15% PLLA, at least about 20% PLLA, at least about 25% PLLA, at least about 30% PLLA, at least about 35% PLLA, at least about 40% PLLA, at least about 45% PLLA, at least about 50% PLLA, at least about 55% PLLA, at least about 60% PLLA, at least about 65% PLLA, at least about 70% PLLA, at least about 75% PLLA, at least about 80% PLLA, at least about 85% PLLA, at least about 90% PLLA, at least about 95% PLLA, at least about 96% PLLA, at least about 97% PLLA, at least about 98% PLLA, at least about 99% PLLA up to about 100% PLLA may be suitable for various methods of the current application.

Various methods of the application comprise the step of collecting lactones produced from the plastic material. Methods of collecting lactones are known in the art. Any method of collecting lactones may be used in the various methods.

Efficient methods of recycling plastic materials comprising PLLA are provided herein. Although plastic materials comprising PLLA are biodegradable, the rate of biodegradation of consumer products comprising PLLA is lengthy, up to 5 years for some medical products. The efficient methods of recycling plastic materials comprising PLLA can be performed in shorter time spans than 5 years. After the plastic comprising PLLA is depolymerized by a modified subtilisin of the current application, the resulting lactones can be synthesized into new materials comprising PLLA. Methods of synthesizing lactones into PLLA are known in the art. Any suitable method for synthesizing lactones into PLLA may be used in the methods of the application.

Methods of regenerating L-lactate monomers are provided herein. Methods of regenerating L-lactate monomers comprise the steps of providing a plastic material comprising PLLA, incubating the plastic material with a modified subtilisin described herein and collecting the L-lactate monomers regenerated from the plastic material. Any method of collecting L-lactate monomers may be used in the methods of the application. It is recognized the step of collecting L-lactate monomers may further comprise a step of separating L-lactate monomers from L-lactate dimers or trimers.

It will be understood that the reference to the below examples is for illustration purposes only and do not limit the scope of the claims.

### EXAMPLES

### Example 1. Cloning, Site Directed Mutagenesis, Bacillus subtilis WB800 Electroporation

The P_{veg} promoter was amplified from Bacillus subtilis WB800⁴⁴ using primers SEQ ID NO:8 and SEQ ID NO:9 (Table 1) to create a strong expression vector. The resulting PCR product was digested using restriction enzymes AatII and EcoRI. The resulting digest was ligated with a correspondingly digested plasmid ECE732 obtained from the Bacillus Genome Stock Center (BGSU) at Ohio State University. Bacillus pumilus B12⁴³ aprE was amplified using primers JC14 and JC83. The resulting product was digested using restriction enzymes SpeI and XbaI and ligated into the newly created ECE732 P_{veg} digested with the same enzymes. Bacillus subtilis PY79 aprE was PCR amplified with primers SEQ ID NOS:9 and 8. Importantly, the GTG start codon of B. subtilis PY79 aprE was changed to ATG during this amplification. This product was also digested with SpeI and XbaI and cloned into ECE732 P_{veg} as described B. pumilus B12 aprE. Forward primers for both enzymes were designed to include a ribosomal binding site (TAAGGAGGTCAAAA) immediately before the start codon.

Site directed mutagenesis was performed by creating 36 bp primers with 5′ overlapping complementary forward and reverse primers (Table 1). PCR reactions were run using LATaq (TaKaRa) with 10 μM PCR primers and 100-150 ng of template DNA. Reaction cycles consisted of a 4-minute denaturation at 94° C. followed by 20 cycles of 94° C. for 30 seconds, 50° C. for 30 seconds, 54° C. for 30 seconds, 68° C. for 15 minutes, and a final cycle of 68° C. for 20 minutes. PCR products were subsequently digested with Dpnl (NEB) at 37° C. overnight to remove template DNA. The resulting digest was subsequently cloned into E. coli DH5α (NEB) and plated on selective media. Plasmid DNA was subsequently miniprepped and sequences to verify mutations.

Plasmid DNA was electroporated into protease-deficient B. subtilis WB800 using the protocol described by Lu et al. (2012)45 with slight modifications. Briefly, single colonies of B. subtilis WB800 were inoculated into 50 mL of LB and incubated overnight at 37° C. with shaking at 250 RPM. One mL of overnight culture was used to inoculate 40 mL of LB+0.5M Sorbitol and incubated under the same incubation conditions for 5-6 hours until an OD₆₀₀ of ^{~}1.5-1.7. Cells were centrifuged and washed four times with ice-cold electroporation buffer⁴⁶ consisting of 0.5M sorbitol, 0.5M mannitol, 10% glycerol and 7.5% glycine betaine. Cells were suspended in electroporation buffer to an OD₆₀₀ of 6.0. At this point, competent cells could be frozen down for later use. Sixty μL of competent cells were mixed with 1 μL of plasmid DNA (50 ng/μL) and electroporated (24 kV/cm, 200 Ω, 25 μF) in a 1-mm electroporation cuvette yielding a time constant of ^{~}5.0-5.2 ms. Cells were recovered in 1 mL of LB containing 0.5M sorbitol and 0.38M mannitol for 3 hours at 37° C. and plated on LB media containing 2 μg/mL erythromycin and 25 μg/mL lincomycin for plasmid selection as well as 10% skim milk for detection of protease activity.

### Example 2. Enzyme Expression and Sample Preparation

B. subtilis WB800 containing ECE732 Pveg were grown in LB+0.01% CaCl₂)*2H2O with 2 μg/mL erythromycin and 25 μg/mL lincomycin for plasmid selection. Ten mL overnights were incubated at 37 C with shaking at 250 RPM for 12 hours. Overnights were back diluted into 50 mL of fresh media at a starting OD₀₀ of 0.01 and incubated for 27 hours. At 27 hours, 30 mL of sample was centrifuged at 20,000 RPM in a Beckman Coulter JA-20 fixed angle rotor. Twenty mL of cell-free supernatant was concentrated using Pierce™ Protein Concentrator PES with a 10 kDa molecular weight cutoff (Fisher Scientific) to a final volume of ^{~}100-250 μL. Twenty mL of ice-cold 100 mM Tris base pH 8.0+1 mM CaCl2 was added to the sample and reconcentrated to ^{~}100-250 μL. This crude, concentrated protein preparation was then diluted to ^{~}600 μL and spun at 13.3k RPM for 20 minutes to remove insoluble materials. This protein preparation was then used for measuring polylactic acid depolymerization activity.

To determine protease concentration in our crude preparation, 1 mL of cell-free supernatant was acid precipitated with 10% (w/v) final concentration of trichloroacetic acid and incubated on ice for several hours. Samples were then spun at 13.3k RPM for 20 minutes to pellet the protein. The sample was then washed 3 times with ice-cold 95% ethanol and resuspended in 100 μL of a solution consisting of 8M urea/2M thiourea. Samples were then run on SDS-PAGE and protein concentration for the protein bands of interest, i.e., subtilisin bands, were quantified using a bovine serum albumin (BSA) standard curve consisting of protein concentrations 4.5, 3.0, 1.5, 0.75, and 0.375 μg BSA. The standard curve and protein bands were quantified using gel analysis tools in ImageJ.

### Example 3. Poly-(L-Lactic) Acid Depolymerization Assay

Glass shell vials (1 dram) (Carolina Biological Supply Company) (Polysciences, Inc.) were used for casting PLLA films. PLLA with an intrinsic viscosity of 1.8 dl/g (Mw ^{~}80-100 kDa) (Polysciences, Inc.) was dissolved at a concentration of 10 mg/mL in chloroform. Dissolved PLLA was added to glass vials at a volume of 1.25 mL and chloroform was allowed to evaporate overnight in a chemical fume hood, creating a solid PLLA film within the vials. One-hundred μg of crude concentrated enzyme preparations in 100 mM Tris buffer pH 8.0 were added to PLLA vials at a final volume of 1 mL. Incubations were performed statically at 30° C. overnight (^{~}15-17 hours). Supernatant was then removed from the PLLA vials and heat inactivated at 95° C. for 5 minutes to inactivate the proteases prior to quantifying the L-lactate using an enzymatic L-lactate assay (described below). All assays were run in triplicate and included a no PLLA control sample to account for background L-lactate.

### Example 4. L-Lactate Assay

Quantification of L-lactate was performed using an enzymatic L-lactate assay with the mechanistic details shown in FIG. 8. Reaction buffer (2.5× stock) consists of 57.5 mM Tris base (pH 7.5), 24.5 mM potassium chloride, 0.045 mM flavin mononucleotide, and 0.002075% bovine serum albumin. Nicotinamide adenine dinucleotide (NAD+) was prepared at a 10× concentration and consists of 30 mM NAD+ dissolved in 20 mM Tris base pH 7.5. Resazurin was prepared at a 20× concentration of 7 mM in ddH2O. Lactate dehydrogenase from rabbit muscle (Millipore Sigma) was prepared by bringing the enzyme to a final concentration of 10 U/μL in a 50/50 mixture of 100 mM phosphate buffer pH 7.0 and 100% glycerol for a final concentration of 50 mM phosphate buffer pH 7.0 and 50% glycerol. Diaphorase from Clostridium kluyveri (Millipore Sigma) was prepared by dissolving the enzyme at a concentration of 0.05 U/μL in a 50/50 mixture of 2.5× reaction buffer and 100% glycerol for a final concentration of 1.75× reaction buffer and 50% glycerol. All stock reagents were stored at −20° C. until use. Reactions were performed in a 96-well plate and consisted of 50 μL of sample with 50 μL of reaction buffer consisting of 40 μL assay buffer, 10 μL NAD+, 5 μL resazurin, 1 μL diaphorase, and 1 μL lactate dehydrogenase. Reactions were carried out for 1 hour in the dark. The assay was measured by determining the fluorescence of the mixture at 530ex/580em and standardized to a standard curve consisting of 1280, 640, 320, 160, 80, 40, 20, 10, 0 μM L-lactate, with smaller values of 6, 3, and 1.5 μM L-lactate also within the limit of detection (FIG. 8). L-lactate concentration was standardized to protein concentration as described above. All data reflects the average of three biological replicates with the L-lactate detected in no PLLA controls subtracted.

### Example 5. Protease Activity

N-succinyl-Ala-Ala-Pro-Phe-p-nitroanilide (Sigma-Aldrich) and N-succinyl-(Ala)₃-p-nitroanilide were dissolved separately in 100 mM Tris Base (pH 8.0) at a concentration of 1.25 mM. Substrate was mixed with crude protease samples at a ratio of 4:1 (80 μL substrate and 20 μL crude supernatant) for a final substrate concentration of 1.0 mM. Assays were run in triplicate in a Cytation 5 Cell Imaging Multi-Mode Reader at 30° C. for 10 minutes with OD₄₁₀ measurements taken every 30 seconds. Beer's law was used to calculate the amount of mM of substrate released per minute under the aforementioned assay conditions. The rate of substrate release in mM/min was standardized to protease concentration using a BSA standard curve as previously described. All results were demonstrated as a percentage standardized to wildtype.

### Example 6 Homology Modeling and in Silico Analysis

An alignment of subtilisin homologs was created by searching for enzymes sharing 50% identity to B. pumilus subtilisin (UniProt primary accession: D3WK97) using the UniRef feature through UniProt. The resulting 582 amino acid sequences were aligned using the MegAlign Pro feature in DNAstar. Consensus sequence logos were also obtained from MegAlign Pro.

The structure of BsAprE and BpAprE was determined through homology modeling with a published crystal structure of the B. subtilis subtilisin (Protein Data Bank accession entry 1SCJ)47, providing an RMSD values of 0.345 Å and 0.479 Å, respectively. Homology modeling was used to account for two non-matching amino acids in the published structure including a cysteine in the catalytic serine 221 residue of the wildtype enzyme. Mutations were generated in the protein by aligning the new sequence containing single amino acid substitutions and generating a new homology model of the protein. Enzymes were imaged in MOE as either ribbon diagrams or as electrostatic potential surface maps using the Active LP setting. Active LP shows regions that are mildly polar (blue), hydrogen bonding (purple), and hydrophobic (green).

### Example 7 Optimization of Reaction Conditions

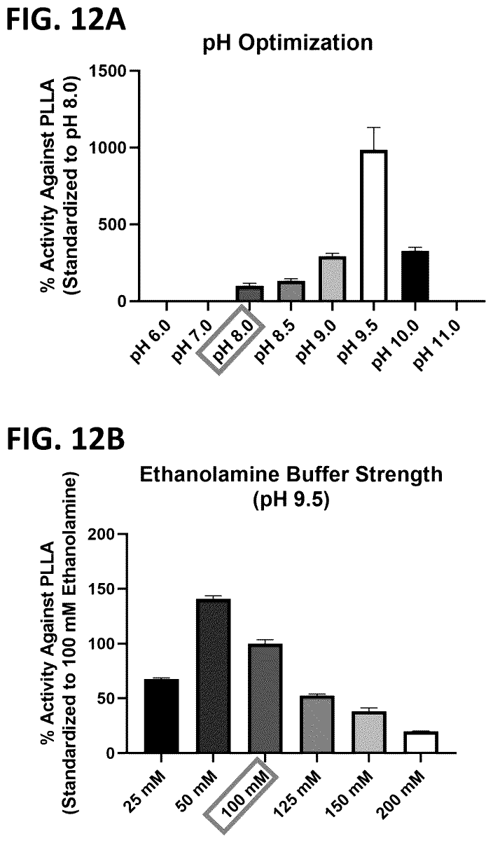

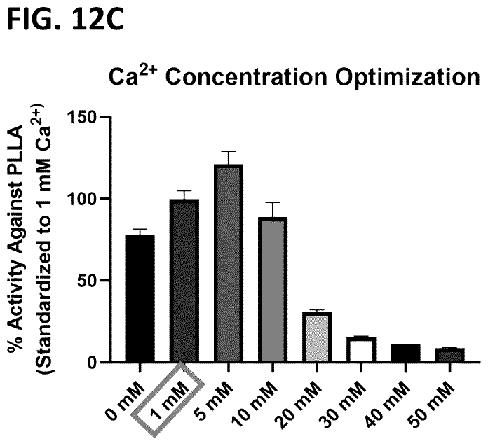

In some experiments, evaluation of enzymatic depolymerization of poly-L-lactic acid (PLLA) was performed in 100 mM Tris buffer (pH 8.0) buffer with 1 mM CaCl₂ and incubation at 30° C. (FIG. 12). Various pH conditions were evaluated such as pH 7.5, 8, 8.5, 9, 9.5, 10 and 10.5. Various buffers including ethanolamine (or borate buffer) were evaluated. Ethanolamine buffer at pH 9.5 resulted in a substantial (approximately 10 fold) increase in activity over previous conditions. The modified subtilisin variants showed activity between pH 8.0 and 10.0. Various concentrations of ethanolamine buffer were evaluated. Using 50 mM ethanolamine buffer showed an additional 40% increase in activity as compared to 100 mM. Various calcium chloride concentrations were evaluated in the 50 mM ethanolamine buffer, pH between about 8 and 10. A calcium chloride (CaCl₂) concentration of 5 mM increased activity an additional 20%.

### Example 8. LC MS Analysis

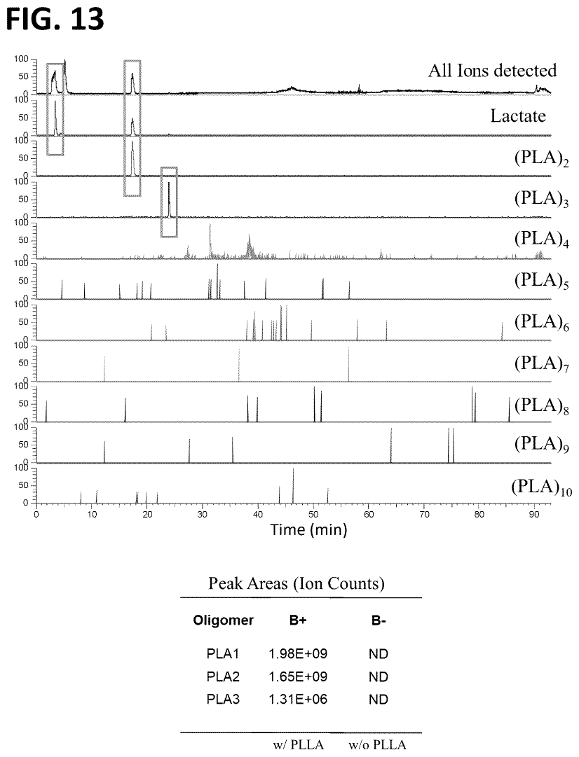

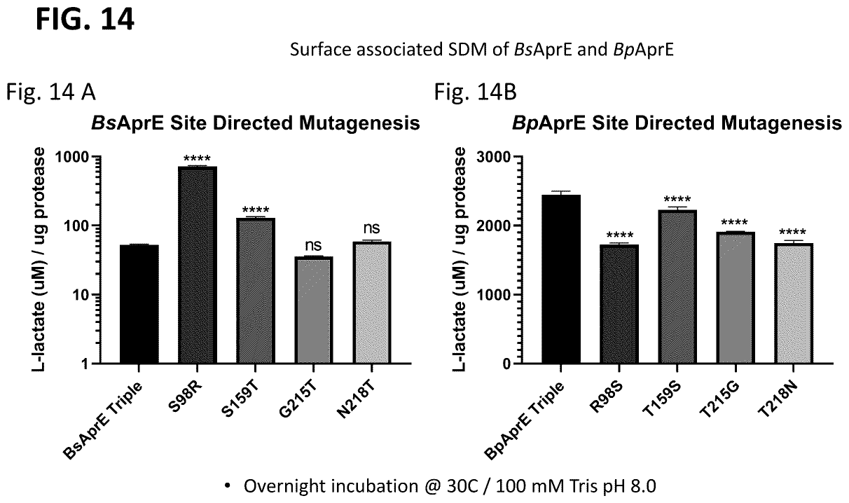

LC-MS analysis was performed on reaction products. The depolymerization assay yields predominantly L-lactate monomers and dimers, with a small amount of trimers also detected (FIG. 13).

### Sequences

The wildtype BpAprE and BsAprE polypeptides are produced with a secretion signal and a pro-peptide sequence. The secretion signal and the pro-peptide sequence are processed during maturation into the functional (mature) polypeptide. The amino acid sequences of the mature forms of the BpAprE and BsAprE polypeptides are provided below herein; the amino acid sequences of the full-length, wild-type BpAprE and BsAprE polypeptides are provided below herein.

Mature BpAprE
SEQ ID NO: 1
AQTVPYGIPQIKAPAVHAQGYKGANVKVAVLDTGIHAAHPDLNVAGG
ASFVPSEPNATQDFQSHGTHVAGTIAALDNTIGVLGVAPSASLYAVK
VLDRYGDGQYSWIISGIEWAVANNMDVINMSLGGPNGSTALKNAVDT
ANNRGVVVVAAAGNSGSTGSTSTVGYPAKYDSTIAVANVNSNNVRNS
SSSAGPELDVSAPGTSILSTVPSSGYTSYTGTSMASPHVAGAAALIL
SKYPNLSTSQVRQRLENTATPLGNSFYYGKGLINVQAASN
Mature BsAprE
SEQ ID NO: 2
AQSVPYGISQIKAPALHSQGYTGSNVKVAVIDSGIDSSHPDLNVRGG
ASFVPSETNPYQDGSSHGTHVAGTIAALNNSIGVLGVAPSASLYAVK
VLDSTGSGQYSWIINGIEWAISNNMDVINMSLGGPTGSTALKTVVDK
AVSSGIVVAAAAGNEGSSGSTSTVGYPAKYPSTIAVGAVNSSNQRAS
FSSAGSELDVMAPGVSIQSTLPGGTYGAYNGTSMATPHVAGAAALIL
SKHPTWTNAQVRDRLESTATYLGNSFYYGKGLINVQAAAQ
Wild-type BpAprE
Bacillus pumilus subtilisin wildtype amino acid
sequence (BpAprE)
SEQ ID NO: 3
MCVKKKNVMTSVLLAVPLLFSAGFGGSIANAETASKSESEKSYIVGF
KASATTNSSKKQAVTQNGGKLEKQYRLINAAQVKMSEQAAKKLEHDP
SIAYVEEDHKAEAYAQTVPYGIPQIKAPAVHAQGYKGANVKVAVLDT
GIHAAHPDLNVAGGASFVPSEPNATQDFQSHGTHVAGTIAALDNTIG
VLGVAPSASLYAVKVLDRYGDGQYSWIISGIEWAVANNMDVINMSLG
GPNGSTALKNAVDTANNRGVVVVAAAGNSGSTGSTSTVGYPAKYDST
IAVANVNSNNVRNSSSSAGPELDVSAPGTSILSTVPSSGYTSYTGTS
MASPHVAGAAALILSKYPNLSTSQVRQRLENTATPLGNSFYYGKGLI
NVQAASN
Wild-type BsAprE
SEQ ID NO: 4
MRSKKLWISLLFALTLIFTMAFSNMSAQAAGKSSTEKKYIVGFKQTM
SAMSSAKKKDVISEKGGKVQKQFKYVNAAAATLDEKAVKELKKDPSV
AYVEEDHIAHEYAQSVPYGISQIKAPALHSQGYTGSNVKVAVIDSGI
DSSHPDLNVRGGASFVPSETNPYQDGSSHGTHVAGTIAALNNSIGVL
GVAPSASLYAVKVLDSTGSGQYSWIINGIEWAISNNMDVINMSLGGP
TGSTALKTVVDKAVSSGIVVAAAAGNEGSSGSTSTVGYPAKYPSTIA
VGAVNSSNQRASFSSAGSELDVMAPGVSIQSTLPGGTYGAYNGTSMA
TPHVAGAAALILSKHPTWTNAQVRDRLESTATYLGNSFYYGKGLINV
QAAAQ

TABLE 1
Primers

| SEQ ID | | | | | |
|---|---|---|---|---|---|
| NO: | | | | | |
| 5 | B12 | AprE | Restriction | R | aaaaaaactagtTTAGTTAGAAGCCGCTTGAA |
| | | | digestion | | |
| | | | (SpeI) | | |
| 6 | B12 | AprE | New forward | F | aaaaaaTCTAGATAAGGAGGTCAAAAATGTGCGTGA |
| | | | w/ RBS and | | AAAAGAAAA |
| | | | new start | | |
| | | | codon | | |
| | | | (XbaI) | | |
| 7 | B12 | AprE | Verify | R | TTAGTTAGAAGCCGCTTGAA |
| | | | insert | | |
| 8 | ECE732 | Pveg | Digestion | F | aaaaaaGACGTCGGAGTTCTGAGAATTGGTATG |
| | | | for new | | |
| | | | promoter | | |
| | | | (AatII) | | |
| 9 | ECE732 | Pveg | Digestion | R | aaaaaaGAATTCACTACATTTATTGTACAACACGAG |
| | | | for new | | |
| | | | promoter | | |
| | | | (EcoRI) | | |
| 10 | ECE732 | Pveg | Screening | F | GGAGTTCTGAGAATTGGTATG |
| | | | insertion | | |
| 11 | ECE732 | Pveg | Screening | R | ACTACATTTATTGTACAACACGAG |
| | | | insertion | | |
| 12 | B. | AprE | Digestion | F | aaaaaaTCTAGATAAGGAGGTCAAAAATGAGAAGCA |
| | subtilis | | for | | AAAAATTGTGG |
| | | | insertion | | |
| | | | into ECE | | |
| | | | plasmid | | |
| | | | (XbaI) | | |
| 13 | B. | AprE | Digestion | R | aaaaaaACTAGTTTATTGTGCAGCTGCTTGTAC |
| | subtilis | | for | | |
| | | | insertion | | |
| | | | into ECE | | |
| | | | plasmid | | |
| | | | (SpeI) | | |
| 14 | ECE732 | ECE732 | Sequencing | F | GAGCGGATACATATTTGAATG |
| | | plasmid | | | |
| 15 | ECE743 | N/A | Plasmid | R | GGATAACAATTTCACACAGG |
| | (TM1844) | | primer | | |
| 16 | B. | AprE | S33T | F | GCTGTTATCGACACAGGAATTGACTCTTCTCATCCT |
| | subtilis | | | | |
| 17 | B. | AprE | S33T | R | AGAGTCAATTCCTGTGTCGATAACAGCTACTTTTAC |
| | subtilis | | | | |
| 18 | B. | AprE | S62Q | F | TACCAGGACGGCCAATCTCACGGTACGCATGTAGCC |
| | subtilis | | | | |
| 19 | B. | AprE | S62Q | R | CGTACCGTGAGATTGGCCGTCCTGGTATGGGTTTGT |
| | subtilis | | | | |
| 20 | B. | AprE | T99Y | F | GTGCTTGATTCATATGGAAGCGGCCAATATAGCTGG |
| | subtilis | | | | |
| 21 | B. | AprE | T99Y | R | TTGGCCGCTTCCATATGAATCAAGCACTTTTACTGC |
| | subtilis | | | | |
| 22 | B. | AprE | S101D | F | GATTCAACAGGAGACGGCCAATATAGCTGGATTATT |
| | subtilis | | | | |
| 23 | B. | AprE | S101D | R | GCTATATTGGCCGTCTCCTGTTGAATCAAGCACTTT |
| | subtilis | | | | |
| 24 | B. | AprE | T130N | F | CTTGGCGGACCTAACGGTTCTACAGCGCTGAAAACA |
| | subtilis | | | | |
| 25 | B. | AprE | T130N | R | CGCTGTAGAACCGTTAGGTCCGCCAAGGCTCATGTT |
| | subtilis | | | | |
| 26 | B. | AprE | E156S | F | GCAGCCGGAAACTCAGGTTCATCCGGAAGCACAAGC |
| | subtilis | | | | |
| 27 | B. | AprE | E156S | R | TCCGGATGAACCTGAGTTTCCGGCTGCGGCAGCAAC |
| | subtilis | | | | |
| 28 | B. | AprE | F189S | F | CAAAGAGCTTCATCATCCAGCGCAGGTTCTGAGCTT |
| | subtilis | | | | |
| 29 | B. | AprE | F189S | R | ACCTGCGCTGGATGATGAAGCTCTTTGGTTGCTGCT |
| | subtilis | | | | |
| 30 | B12 | AprE | T33S | F | GCTGTCCTTGATAGCGGAATCCACGCCGCACACCCT |
| 31 | B12 | AprE | T33S | R | GGCGTGGATTCCGCTATCAAGGACAGCTACTTTGAC |
| 32 | B12 | AprE | Q62S | F | ACACAAGACTTTTCATCACACGGAACTCACGTAGCC |
| 33 | B12 | AprE | Q62S | R | AGTTCCGTGTGATGAAAAGTCTTGTGTGGCATTTGG |
| 34 | B12 | AprE | Y99T | F | GTATTAGATCGTACAGGCGATGGACAATACAGCTGG |
| 35 | B12 | AprE | Y99T | R | TTGTCCATCGCCTGTACGATCTAATACTTTAACGGC |
| 36 | B12 | AprE | D101S | F | GATCGTTACGGCAGCGGACAATACAGCTGGATTATC |
| 37 | B12 | AprE | D101S | R | GCTGTATTGTCCGCTGCCGTAACGATCTAATACTTT |
| 38 | B12 | AprE | N130T | F | TTAGGCGGACCAACAGGCTCCACAGCGCTTAAAAAT |
| 39 | B12 | AprE | N130T | R | CGCTGTGGAGCCTGTTGGTCCGCCTAAGCTCATATT |
| 40 | B12 | AprE | S156E | F | GCAGCAGGGAATGAAGGTTCAACTGGCTCTACTAGC |
| 41 | B12 | AprE | S156E | R | GCCAGTTGAACCTTCATTCCCTGCTGCGGCAACGAC |
| 42 | B12 | AprE | S189F | F | GTCAGAAACTCGTTTTCTAGCGCAGGTCCTGAATTA |
| 43 | B12 | AprE | S189F | R | ACCTGCGCTAGAAAACGAGTTTCTGACATTGTTGCT |
| 44 | B12 | B12 | P129A | F | AGCTTAGGCGGAGCAAACGGCTCCACAGCGCTTAAA |
| | | AprE | | | |
| 45 | B12 | B12 | P129A | R | TGTGGAGCCGTTTGCTCCGCCTAAGCTCATATTGAT |
| | | AprE | | | |
| 46 | B12 | B12 | P129A | F | AGCTTAGGCGGAGCAACAGGCTCCACAGCGCTTAAA |
| | | AprE | (w/ N130T | | |
| | | | compatability) | | |
| 47 | B12 | B12 | P129A | R | TGTGGAGCCTGTTGCTCCGCCTAAGCTCATATTGAT |
| | | AprE | (w/ N130T | | |
| | | | compatability) | | |
| 48 | B. | AprE | T159F | F | AATTCAGGTTCATTTGGCTCTACTAGCACCGTTGGC |
| | pumilus | Triple | | | |
| 49 | B. | AprE | T159F | R | GCTAGTAGAGCCAAATGAACCTGAATTCCCTGCTGC |
| | pumilus | Triple | | | |
| 50 | B. | AprE | T162F | F | TCAACTGGCTCTTTTAGCACCGTTGGCTATCCTGCA |
| | pumilus | Triple | | | |
| 51 | B. | AprE | T162F | R | GCCAACGGTGCTAAAAGAGCCAGTTGAACCTGAATT |
| | pumilus | Triple | | | |
| 52 | B. | AprE | R98S | F | AAAGTATTAGATTCATACGGCAGCGGACAATACAGC |
| | pumilus | Triple | | | |
| 53 | B. | AprE | R98S | R | TCCGCTGCCGTATGAATCTAATACTTTAACGGCATA |
| | pumilus | Triple | | | |
| 54 | B. | AprE | Y99F | F | GTATTAGATCGTTTTGGCAGCGGACAATACAGCTGG |
| | pumilus | Triple | | | |
| 55 | B. | AprE | Y99F | R | TTGTCCGCTGCCAAAACGATCTAATACTTTAACGGC |
| | pumilus | Triple | | | |
| 56 | B. | AprE | Y99W | F | GTATTAGATCGTTGGGGCAGCGGACAATACAGCTGG |
| | pumilus | Triple | | | |
| 57 | B. | AprE | Y99W | R | TTGTCCGCTGCCCCAACGATCTAATACTTTAACGGC |
| | pumilus | Triple | | | |
| 58 | B. | AprE | T215A | F | AGCAGTGGATATGCATCTTATACGGGAACATCGATG |
| | pumilus | Triple | | | |
| 59 | B. | AprE | T215A | R | TCCCGTATAAGATGCATATCCACTGCTTGGTACTGT |
| | pumilus | Triple | | | |
| 60 | B. | AprE | T215G | F | AGCAGTGGATATGGCTCTTATACGGGAACATCGATG |
| | pumilus | Triple | | | |
| 61 | B. | AprE | T215G | R | TCCCGTATAAGAGCCATATCCACTGCTTGGTACTGT |
| | pumilus | Triple | | | |
| 62 | B. | AprE | Q103S | F | TACGGCAGCGGATCATACAGCTGGATTATCAGCGGT |
| | pumilus | Triple | | | |
| 63 | B. | AprE | Q103S | R | AATCCAGCTGTATGATCCGCTGCCGTAACGATCTAA |
| | pumilus | Triple | | | |
| 64 | B. | AprE | Q103Y | F | TACGGCAGCGGATATTACAGCTGGATTATCAGCGGT |
| | pumilus | Triple | | | |
| 65 | B. | AprE | Q103Y | R | AATCCAGCTGTAATATCCGCTGCCGTAACGATCTAA |
| | pumilus | Triple | | | |
| 66 | B. | AprE | T130S | F | TTAGGCGGACCATCAGGCTCCACAGCGCTTAAAAAT |
| | pumilus | Triple | | | |
| 67 | B. | AprE | T130S | R | CGCTGTGGAGCCTGATGGTCCGCCTAAGCTCATATT |
| | pumilus | Triple | | | |
| 68 | B. | AprE | Y104F | F | GGCAGCGGACAATTTAGCTGGATTATCAGCGGTATT |
| | pumilus | Triple | | | |
| 69 | B. | AprE | Y104F | R | GATAATCCAGCTAAATTGTCCGCTGCCGTAACGATC |
| | pumilus | Triple | | | |
| 70 | B. | AprE | Y104W | F | GGCAGCGGACAATGGAGCTGGATTATCAGCGGTATT |
| | pumilus | Triple | | | |
| 71 | B. | AprE | Y104W | R | GATAATCCAGCTCCATTGTCCGCTGCCGTAACGATC |
| | pumilus | Triple | | | |
| 72 | B. | AprE | P129S | F | AGCTTAGGCGGATCAACAGGCTCCACAGCGCTTAAA |
| | pumilus | Triple | | | |
| 73 | B. | AprE | P129S | R | TGTGGAGCCTGTTGATCCGCCTAAGCTCATATTGAT |
| | pumilus | Triple | | | |
| 74 | B. | AprE | T218S | F | TATACATCTTATTCAGGAACATCGATGGCATCTCCT |
| | pumilus | Triple | | | |
| 75 | B. | AprE | T218S | R | CATCGATGTTCCTGAATAAGATGTATATCCACTGCT |
| | pumilus | Triple | | | |
| 76 | B. | AprE | Y217F | F | GGATATACATCTTTTACGGGAACATCGATGGCATCT |
| | pumilus | Triple | | | |
| 77 | B. | AprE | Y217F | R | CGATGTTCCCGTAAAAGATGTATATCCACTGCTTGG |
| | pumilus | Triple | | | |
| 78 | B. | AprE | Y217L | F | GGATATACATCTCTGACGGGAACATCGATGGCATCT |
| | pumilus | Triple | | | |
| 79 | B. | AprE | Y217L | R | CGATGTTCCCGTCAGAGATGTATATCCACTGCTTGG |
| | pumilus | Triple | | | |
| 80 | B. | BsAprE | N218T | F | TACGGCGCTTATACAGGAACGTCCATGGCGACTCCT |
| | subtilis | | | | |
| 81 | B. | BsAprE | N218T | R | CATGGACGTTCCTGTATAAGCGCCGTAAGTGCCTCC |
| | subtilis | | | | |
| 82 | B. | BpAprE | T218N | F | TATACATCTTATAATGGAACATCGATGGCATCTCCT |
| | pumilus | | | | |
| 83 | B. | BpAprE | T218N | R | CATCGATGTTCCATTATAAGATGTATATCCACTGCT |
| | pumilus | | | | |
| 84 | B. | BsAprE | N218T | F | TACGGCGCTTATACAGGAACGTCCATGGCGACTCCT |
| | subtilis | | | | |
| 85 | B. | BsAprE | N218T | R | CATGGACGTTCCTGTATAAGCGCCGTAAGTGCCTCC |
| | subtilis | | | | |
| 86 | B. | BpAprE | T218N | F | TATACATCTTATAATGGAACATCGATGGCATCTCCT |
| | pumilus | | | | |
| 87 | B. | BpAprE | T218N | R | CATCGATGTTCCATTATAAGATGTATATCCACTGCT |
| | pumilus | | | | |
| 88 | B. | BsAprE | S159T | F | AACTCAGGTTCAACAGGAAGCACAAGCACAGTCGGC |
| | subtilis | | | | |
| 89 | B. | BsAprE | S159T | R | GCTTGTGCTTCCTGTTGAACCTGAGTTTCCGGCTGC |
| | subtilis | | | | |
| 90 | B. | BsAprE | G215T | F | GGAGGCACTTACACAGCTTATAACGGAACGTCCATG |
| | subtilis | | | | |
| 91 | B. | BsAprE | G215T | R | TCCGTTATAAGCTGTGTAAGTGCCTCCAGGAAGTGT |
| | subtilis | | | | |
| 92 | B. | BsAprE | S98R | F | AAAGTGCTTGATAGATATGGAAGCGGCCAATATAGC |
| | subtilis | | (Re-design) | | |
| 93 | B. | BsAprE | S98R | R | GCCGCTTCCATATCTATCAAGCACTTTTACTGCATA |
| | subtilis | | (Re-design) | | |
| 94 | B. | BpAprE | T159S | F | AATTCAGGTTCATCAGGCTCTACTAGCACCGTTGGC |
| | pumilus | | | | |
| 95 | B. | BpAprE | T159S | R | GCTAGTAGAGCCTGATGAACCTGAATTCCCTGCTGC |
| | pumilus | | | | |
| 96 | B. | BsAprE | A187N | F | AGCAACCAAAGAAATTCATTCTCCAGCGCAGGTTCT |
| | subtilis | | | | |
| 97 | B. | BsAprE | A187N | R | GCTGGAGAATGAATTTCTTTGGTTGCTGCTGTTTAC |
| | subtilis | | | | |

1. PlasticsEurope 2011. Plastics—the Facts 2011 An analysis of European plastics production, demand and recovery for 2010. PlasticsEurope https://www.plasticseurope.org/en/resources/publications/115-plastics-facts-2011.
2. Plastics the Facts 2021 ⋅ Plastics Europe. Plastics Europe https://plasticseurope.org/knowledge-hub/plastics-the-facts-2021/.
3. Danso, D., Chow, J. & Streit, W. R. Plastics: Environmental and Biotechnological Perspectives on Microbial Degradation. Appl. Environ. Microbiol. 85, (2019).
4. Jambeck, J. R. et al. Plastic waste inputs from land into the ocean. Science 347, 768-771 (2015).
5. Barnes, D. K. A., Galgani, F., Thompson, R. C. & Barlaz, M. Accumulation and fragmentation of plastic debris in global environments. Phil. Trans. R. Soc. B 364, 1985-1998 (2009).
6. Zhang, S. et al. Microplastics in the environment: A review of analytical methods, distribution, and biological effects. TrAC Trends in Analytical Chemistry 111, 62-72 (2019).
7. Gross, R. A. & Kalra, B. Biodegradable Polymers for the Environment. Science 297, 803-807 (2002).
8. Flieger, M., Kantorová, M., Prell, A., Řezanka, T. & Votruba, J. Biodegradable plastics from renewable sources. Folia Microbiol 48, 27 (2003).
9. Auras, R., Harte, B. & Selke, S. An overview of polylactides as packaging materials. Macromol Biosci 4, 835-864 (2004).
10. Haider, T. P., Völker, C., Kramm, J., Landfester, K. & Wurm, F. R. Plastics of the Future? The Impact of Biodegradable Polymers on the Environment and on Society. Angewandte Chemie International Edition 58, 50-62 (2019).
11. Iwata, T. Biodegradable and Bio-Based Polymers: Future Prospects of Eco-Friendly Plastics. Angewandte Chemie International Edition 54, 3210-3215 (2015).
12. Wei, R. & Zimmermann, W. Microbial enzymes for the recycling of recalcitrant petroleum-based plastics: how far are we? Microbial Biotechnology 10, 1308-1322 (2017).
13. EUBIO_Admin. Market 2021. European Bioplastics e.V. https://www.european-bioplastics.org/market/.
14. Nishida, H. & Tokiwa, Y. Distribution of poly(β-hydroxybutyrate) and poly(ε-caprolactone)aerobic degrading microorganisms in different environments. J Environ Polym Degr 1, 227-233 (1993).
15. Pranamuda, H., Tokiwa, Y. & Tanaka, H. Polylactide Degradation by an Amycolatopsis sp. Applied and environmental microbiology 63, 1637-1640 (1997).
16. Madhavan Nampoothiri, K., Nair, N. R. & John, R. P. An overview of the recent developments in polylactide (PLA) research. Bioresource Technology 101, 8493-8501 (2010).
17. Dorgan, J. R., Lehermeier, H. & Mang, M. Thermal and Rheological Properties of Commercial-Grade Poly(Lactic Acid)s. Journal of Polymers and the Environment 8, 1-9 (2000).
18. Henton, D. E., Gruber, P., Lunt, J. & Randall, J. Polylactic Acid Technology. 52.
19. Karamanlioglu, M., Preziosi, R. & Robson, G. D. Abiotic and biotic environmental degradation of the bioplastic polymer poly(lactic acid): A review. Polymer Degradation and Stability 137, 122-130 (2017).
20. Tokiwa, Y. & Calabia, B. P. Biodegradability and biodegradation of poly(lactide). Appl Microbiol Biotechnol 72, 244-251 (2006).
21. Payne, J., McKeown, P. & Jones, M. D. A circular economy approach to plastic waste. Polymer Degradation and Stability 165, 170-181 (2019).
22. Piemonte, V., Sabatini, S. & Gironi, F. Chemical Recycling of PLA: A Great Opportunity Towards the Sustainable Development? J Polym Environ 21, 640-647 (2013).
23. Niaounakis, M. Recycling of biopolymers—The patent perspective. European Polymer Journal 114, 464-475 (2019).
24. Hajighasemi, M. et al. Screening and Characterization of Novel Polyesterases from Environmental Metagenomes with High Hydrolytic Activity against Synthetic Polyesters. Environ. Sci. Technol. 52, 12388-12401 (2018).
25. Qi, X., Ren, Y. & Wang, X. New advances in the biodegradation of Poly(lactic) acid. International Biodeterioration & Biodegradation 117, 215-223 (2017).
26. Kawai, F. et al. Different enantioselectivity of two types of poly(lactic acid) depolymerases toward poly(l-lactic acid) and poly(d-lactic acid). Polymer Degradation and Stability 96, 1342-1348 (2011).
27. Sakai, K., Kawano, H., Iwami, A., Nakamura, M. & Moriguchi, M. Isolation of a thermophilic poly-l-lactide degrading bacterium from compost and its enzymatic characterization. Journal of Bioscience and Bioengineering 92, 298-300 (2001).
28. Zhu, B., Wang, D. & Wei, N. Enzyme Discovery and Engineering for Sustainable Plastic Recycling. Trends in Biotechnology S0167779921000408 (2021) doi:10.1016/j.tibtech.2021.02.008.
29. Zhu et al.—2021—Enzyme Discovery and Engineering for Sustainable P.pdf.
30. Hanphakphoom, S., Maneewong, N., Sukkhum, S., Tokuyama, S. & Kitpreechavanich, V. Characterization of poly(L-lactide)-degrading enzyme produced by thermophilic filamentous bacteria Laceyella sacchari LP175. The Journal of General and Applied Microbiology 60, 13-22 (2014).
31. Lim, H.-A., Raku, T. & Tokiwa, Y. Hydrolysis of polyesters by serine proteases. Biotechnol. Lett. 27, 459-464 (2005).
32. Oda, Y., Yonetsu, A., Urakami, T. & Tonomura, K. Degradation of Polylactide by Commercial Proteases. Journal of Polymers and the Environment 8, 29-32 (2000).
33. Vichaibun, V. & Chulavatnatol, M. A New Assay for the Enzymatic Degradation of Polylactic Acid. 4 (2003).
34. Nakamura, K., Tomita, T., Abe, N. & Kamio, Y. Purification and Characterization of an Extracellular Poly(l-Lactic Acid) Depolymerase from a Soil Isolate, Amycolatopsis sp. Strain K104-1. Appl. Environ. Microbiol. 67, 345-353 (2001).
35. Sukkhum, S., Tokuyama, S., Tamura, T. & Kitpreechavanich, V. A novel poly (L-lactide) degrading actinomycetes isolated from Thai forest soil, phylogenic relationship and the enzyme characterization. J. Gen. Appl. Microbiol. 55, 459-467 (2009).
36. Jarerat, A. & Tokiwa, Y. Degradation of Poly(L-lactide) by a Fungus. Macromolecular Bioscience 1, 136-140 (2001).
37. Williams, D. F. Enzymic Hydrolysis of Polylactic Acid. Engineering in Medicine 10, 5-7 (1981).
38. Contesini, F. J., Melo, R. R. de & Sato, H. H. An overview of Bacillus proteases: from production to application. Critical Reviews in Biotechnology 38, 321-334 (2018).
39. Schallmey, M., Singh, A. & Ward, O. P. Developments in the use of Bacillus species for industrial production. Can. J. Microbiol. 50, 1-17 (2004).
40. Harwood, C. R. & Cranenburgh, R. Bacillus protein secretion: an unfolding story. Trends in Microbiology 16, 73-79 (2008).
41. van Dijl, J. & Hecker, M. Bacillus subtilis: from soil bacterium to super-secreting cell factory. Microbial Cell Factories 12, 3 (2013).
42. Ling Lin Fu et al. Protein secretion pathways in Bacillus subtilis: Implication for optimization of heterologous protein secretion. Biotechnology Advances 25, 1-12 (2007).
43. Bonifer, K. S. et al. Bacillus pumilus B12 Degrades Polylactic Acid and Degradation Is Affected by Changing Nutrient Conditions. Front. Microbiol. 10, (2019).
44. Wu, S.-C. et al. Functional Production and Characterization of a Fibrin-Specific Single-Chain Antibody Fragment from Bacillus subtilis: Effects of Molecular Chaperones and a Wall-Bound Protease on Antibody Fragment Production. AEM 68, 3261-3269 (2002).
45. Lu, Y.-P., Zhang, C., Lv, F. X., Bie, X. M. & Lu, Z.-X. Study on the electro-transformation conditions of improving transformation efficiency for Bacillus subtilis. Letters in Applied Microbiology 55, 9-14 (2012).
46. Meddeb-Mouelhi, F., Dulcey, C. & Beauregard, M. High transformation efficiency of Bacillus subtilis with integrative DNA using glycine betaine as osmoprotectant. Analytical Biochemistry 424, 127-129 (2012).
47. Jain, S. C., Shinde, U., Li, Y., Inouye, M. & Berman, H. M. The crystal structure of an autoprocessed Ser221Cys-subtilisin E-propeptide complex at 2.0 å resolution11 Edited by I. A. Wilson. Journal of Molecular Biology 284, 137-144 (1998).
48. Wang, Z., Wang, Y., Guo, Z., Li, F. & Chen, S. Purification and characterization of poly(L-lactic acid) depolymerase from Pseudomonas sp. strain DS04-T. Polymer Engineering & Science 51, 454-459 (2011).
49. Li, F., Wang, S., Liu, W. & Chen, G. Purification and characterization of poly(l-lactic acid)-degrading enzymes from Amycolatopsis orientalis ssp. orientalis. FEMS Microbiol Lett 282, 52-58 (2008).
50. Akutsu-Shigeno, Y. et al. Cloning and Sequencing of a Poly(dl-Lactic Acid) Depolymerase Gene from Paenibacillus amylolyticus Strain TB-13 and Its Functional Expression in Escherichia coli. Appl Environ Microbiol 69, 2498-2504 (2003).
51. Nakamura, K., Tomita, T., Abe, N. & Kamio, Y. Purification and Characterization of an Extracellular Poly(l-Lactic Acid) Depolymerase from a Soil Isolate, Amycolatopsis sp. Strain K104-1. Appl Environ Microbiol 67, 345-353 (2001).
52. Shah, A. A., Hasan, F., Hameed, A. & Ahmed, S. Biological degradation of plastics: A comprehensive review. Biotechnology Advances 26, 246-265 (2008).
53. Hajighasemi, M. et al. Biochemical and Structural Insights into Enzymatic Depolymerization of Polylactic Acid and Other Polyesters by Microbial Carboxylesterases. Biomacromolecules 17, 2027-2039 (2016).
54. Babson, A. L. & Phillips, G. E. A rapid colorimetric assay for serum lactic dehyurogenase. Clinica Chimica Acta 12, 210-215 (1965).
55. Shapiro, F. & Silanikove, N. Rapid and accurate determination of d- and l-lactate, lactose and galactose by enzymatic reactions coupled to formation of a fluorochromophore: Applications in food quality control. Food Chemistry 119, 829-833 (2010).
56. Williams, D. F. Enzymic Hydrolysis of Polylactic Acid. Engineering in Medicine 10, 5-7 (1981).
57. Kawamura, F. & Doi, R. H. Construction of a Bacillus subtilis double mutant deficient in extracellular alkaline and neutral proteases. J Bacteriol 160, 442-444 (1984).
58. Siezen, R. J. & Leunissen, J. A. Subtilases: the superfamily of subtilisin-like serine proteases. Protein Sci 6, 501-523 (1997).
59. Babé, LiliaM. & Schmidt, B. Purification and biochemical analysis of WprA, a 52-kDa serine protease secreted by B. subtilis as an active complex with its 23-kDa propeptide. Biochimica et Biophysica Acta (BBA)—Protein Structure and Molecular Enzymology 1386, 211-219 (1998).
60. Brode, P. F. et al. Subtilisin BPN′ Variants: Increased Hydrolytic Activity on Surface-Bound Substrates via Decreased Surface Activity. Biochemistry 35, 3162-3169 (1996).
61. Sukkhum, S., Tokuyama, S., Tamura, T. & Kitpreechavanich, V. A novel poly (L-lactide) degrading actinomycetes isolated from Thai forest soil, phylogenic relationship and the enzyme characterization. J Gen Appl Microbiol 55, 459-467 (2009).
62. Jarerat, A., Pranamuda, H. & Tokiwa, Y. Poly(L-lactide)-Degrading Activity in Various Actinomycetes. Macromolecular Bioscience 2, 420-428 (2002).
63. Jarerat, A. & Tokiwa, Y. Degradation of Poly(L-lactide) by a Fungus. Macromolecular Bioscience 1, 136-140 (2001).
64. Jarerat, A., Tokiwa, Y. & Tanaka, H. Microbial Poly(L-Lactide)-Degrading Enzyme Induced by Amino Acids, Peptides, and Poly(L-Amino Acids). Journal of Polymers and the Environment 12, 139-146 (2004).
65. Pranamuda, H. & Tokiwa, Y. Degradation of poly(L-lactide) by strains belonging to genus Amycolatopsis. Biotechnology Letters 21, 901-905 (1999).
66. Austin, H. P. et al. Characterization and engineering of a plastic-degrading aromatic polyesterase. PNAS 115, E4350-E4357 (2018).
67. Yoshida, S. et al. A bacterium that degrades and assimilates poly(ethylene terephthalate). Science 351, 1196-1199 (2016).
68. Akutsu, Y., Nakajima-Kambe, T., Nomura, N. & Nakahara, T. Purification and Properties of a Polyester Polyurethane-Degrading Enzyme from Comamonas acidovorans TB-35. Appl Environ Microbiol 64, 62-67 (1998).
69. Mukai, K., Yamada, K. & Doi, Y. Kinetics and mechanism of heterogeneous hydrolysis of poly[(R)-3-hydroxybutyrate]film by PHA depolymerases. International Journal of Biological Macromolecules 15, 361-366 (1993).
70. Ohtaki, S. et al. Novel Hydrophobic Surface Binding Protein, HsbA, Produced by Aspergillus oryzae. Appl Environ Microbiol 72, 2407-2413 (2006).

1. A modified subtilisin polypeptide comprising at least one amino acid substitution at a position selected from S33, T33, S62, Q62, S98, R98, T99, Y99, D101, S101, Q103, Y104, P129, N130, T130, E156, S156, T159, S159, T162, A187, S189, F189, T215, G215, Y217, N218, and T218 of a subtilisin polypeptide comprising an amino acid sequence selected from the group of subtilisin amino acid sequences set forth in SEQ ID NO:1 and SEQ ID NO:2 and combinations thereof.

2. The modified subtilisin polypeptide of claim 1 wherein the at least one amino acid substitution is selected from T33, Q62, R98, Y99, D101, Q103, Y104, P129, N130, S156, T159, T162, S189, T215, Y217, T218 of a subtilisin polypeptide comprising an amino acid sequence set forth in SEQ ID NO:1.

3. The modified subtilisin polypeptide of claim 1, wherein the at least one amino acid substitution is selected from S33, S62, S98, T99, S101, T130, E156, S159, A187, F189, G215 and N218 of a subtilisin polypeptide comprising an amino acid sequence having the sequence set forth in SEQ ID NO:2.

4. The modified subtilisin polypeptide of claim 1, wherein the amino acid substitution is selected from the group consisting of S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N.

5.-6. (canceled)

7. The modified subtilisin polypeptide of claim 1, comprising at least two amino acid substitutions at a position selected from S33, T33, S62, Q62, S98, R98, T99, Y99, D101, S101, Q103, Y104, P129, N130, T130, E156, S156, T159, S159, T162, A187, S189, F189, T215, G215, Y217, N218, T218 of a subtilisin polypeptide comprising an amino acid sequence selected from the group of subtilisin amino acid sequences set forth in SEQ ID NO:1 and SEQ ID NO:2 and combinations thereof.

8. The modified subtilisin polypeptide of claim 1, comprising at least three amino acid substitutions at a position selected from S33, T33, S62, Q62, S98, R98, T99, Y99, D101, S101, Q103, Y104, P129, N130, T130, E156, S156, T159, S159, T162, A187, S189, F189, T215, G215, Y217, N218, T218 of a subtilisin polypeptide comprising an amino acid sequence selected from the group of subtilisin amino acid sequences set forth in SEQ ID NO:1 and SEQ ID NO:2 and combinations thereof.

9. (canceled)

10. The modified subtilisin polypeptide of claim 1, comprising a combination of amino acid substitutions wherein the combination is selected from the group of combinations of amino acid substitutions consisting of:
(a) D101S, N130T and S189F;
(b) D101A, N130S and S189F;
(c) D101S, N130S and S189F;
(d) D101A, N130T and S189F;
(e) D101S, N130T, S189F and Y217L;
(f) S98R, S159T, N218T and A187N;
(g) S33T, T99Y and E156S;
(h) S159T and N218T;
(i) S98R and N218T;
(j) S33T, S98R, T99Y, E156S, S159T, and N218T;
(k) S33T, S98R, T99Y, E156S, S159T, A187N, and N218T;
(l) S33T and T99Y;
(m) S33T and E156S;
(n) S33T, T99Y and E156S;
(o) T99Y and E156S;
(p) D101S and N130T;
(q) D101A, N130T, S189F and Y217L;
(r) D101S, N130S, S189F and Y217L;
(s) D101S, N130T, S189F and Y217L;
(t) S98R and S159T;
(u) N130T and S189F; and combinations thereof.

11.-12. (canceled)

13. The modified subtilisin polypeptide of claim 10, and further comprising at least one additional mutation selected from the group consisting of: S33T, T33S, S62Q Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N.

14. The modified subtilisin polypeptide of claim 1, wherein the amino acid sequence comprises a combination of amino acid substitutions selected from the group consisting of:
(a) S98R, S159T, N218T and A187N;
(b) S33T, S98R, T99Y, E156S, S159T, and N218T;
(c) D101S, N130T, S189F and Y217L;
(d) D101A, N130T, S189F and Y217L;
(e) D101S, N130S, S189F and Y217L;
(f) D101A, N130S, S189F and Y217L; and
(g) S33T, S98R, T99Y, E156S, S159T, A187N, and N218T.

15. The modified subtilisin polypeptide of claim 14 and further comprising at least one additional mutation selected from the group consisting of: S33T, T33S, S62Q, Q62S, S98R, R98S, T99Y, Y99F, Y99T, Y99W, D101S, D101A, S101D, Q103S, Y104W, Y104F, P129A, P129S, N130T, N130S, T130N, E156S, S156A, S156E, S159T, T159S, T159F, T162F, A187N, S189F, F189S, G215T, T215G, T215A, Y217L, Y217F, N218T, T218S and T218N.

16. The modified subtilisin polypeptide of claim 1, wherein the polypeptide exhibits an an altered activity selected from the group consisting of an altered polylactic acid (PLLA) depolymerization activity, an increased PLLA depolymerization activity, increased PLLA depolymerization activity is at least 1.5× greater than the PLLA depolymerization activity of a subtilisin polypeptide having an amino acid sequence set forth in SEQ ID NO:1 or SEQ ID NO:2, increased PLLA depolymerization activity is at least 1.5× greater than the PLLA depolymerization activity of a subtilisin polypeptide having the amino acid sequence set forth in SEQ ID NO:1, and increased PLLA depolymerization activity is at least 5× greater than the PLLA depolymerization activity of a subtilisin polypeptide having the amino acid sequence set forth in SEQ ID NO:2.

17.-23. (canceled)

24. The modified subtilisin polypeptide of claim 1, wherein the polypeptide exhibits an activity selected from the group comprising an altered protease activity, an altered polylactic acid depolymerization activity, both an altered polylactic acid depolymerization activity and an altered protease activity, an increased yield of soluble L Lactate products, rapid depolymerization activity, depolymerization in less than 18 hours, depolymerization activity at a reaction temperature in the range of about 30° C., rapid depolymerization activity at a low reaction temperature, PLLA depolymerization activity at a pH between about 7 and about 10.5, increased PLLA depolymerization activity at approximately pH 9.5, and increased PLLA depolymerization activity in the presence of between about 1 mM and 10 mM calcium salt.

25.-34. (canceled)

35. A method of quantifying poly (L) lactic acid depolymerization comprising the steps of:
(a) Providing a plastic material comprising PLLA;
(b) Combining an enzyme of interest wherein the enzyme of interest is a modified subtilisin polypeptide of claim 1 with the plastic material comprising PLLA;
(c) Incubating the plastic material comprising PLLA and enzyme of interest at a predetermined temperature;
(d) Collecting the supernatant;
(e) Inactivating the enzyme of interest;
(f) Adding NAD+, L-lactate dehydrogenase, diaphorase and a reactive indicator to the supernatant;
(g) Incubating a mixture comprising supernatant, NAD+, L-lactate dehydrogenase, diaphorase and a reactive indicator;
(h) detecting the reactive indicator;
(i) determining the amount of L-lactate present, and
(j) quantifying the poly(L) lactic acid depolymerization from the amount of L-lactate present.

36. The method of claim 35 wherein NAD+ and L-lactate dehydrogenase are added in excess.

37.-41. (canceled)

42. The method of claim 35, wherein the method has a minimum detection limit of about 1.5 μM lactate.

43. The method of claim 35, wherein the method has a maximum detection limit selected from the group comprising (a) near the saturating concentration of resorufin and (b) wherein the method has a maximum detection limit of about 1.28 mM lactate.

44. (canceled)

45. A method of depolymerizing PLLA comprising the steps of (a) providing a plastic material comprising PLLA and (b) incubating the plastic material with a modified subtilisin polypeptide of claim 1.

46. A method of producing lactones from plastic comprising PLLA, comprising the steps of (a) providing a plastic material comprising PLLA; (b) incubating the plastic material with a modified subtilisin of claim 1; and (c) collecting the lactones produced from the plastic material.

47. An efficient method of recycling plastic material comprising PLLA comprising the steps of (a) providing a plastic material comprising PLLA; (b) incubating the plastic material with a modified subtilisin of claim 1; (c) collecting the lactones produced from the plastic material; and (d) synthesizing the lactones into PLLA.

48. A method of regenerating l-lactate monomers, comprising the steps of (a) providing a plastic material comprising PLLA; (b) incubating the plastic material with a modified subtilisin of claim 1; (c) collecting the I-lactate monomers regenerated from the plastic material.

49. The method of claim 35 wherein the step of incubating the plastic with a modified subtilisin occurs at a predetermined temperature, optionally wherein the predetermined temperature is between about 25° C. and about 35° C., or optionally about 30° C.

50. The method of claim 35, wherein the plastic comprises at least 0.1% PLLA.

51. (canceled)

52. The method of claim 35, comprising the step of adjusting the pH to between about 7 and about 10.5.

53. (canceled)

54. The method of claim 35, comprising adding calcium salt, optionally wherein the calcium salt is calcium chloride.

55.-57. (canceled)


SEQUENCE LISTING (97 sequences)

SEQ ID NO: 1 - protein, 275 aa, Bacillus pumilus
  REGION 1..275: note = misc_feature - Mature BpAprE
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKNAVDTANNRGVVVVAAAGNSGSTGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGNSFYYGKGLINVQAASN

SEQ ID NO: 2 - protein, 275 aa, Bacillus pumilus
  REGION 1..275: note = misc_feature - Mature BsAprE
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 3 - protein, 383 aa, Bacillus pumilus
  REGION 1..383: note = misc_feature - Wild-type BpAprE
MCVKKKNVMTSVLLAVPLLFSAGFGGSIANAETASKSESEKSYIVGFKASATTNSSKKQAVTQNGGKLEKQYRLINAAQVKMSEQAAKKLEHDPSIAYVEEDHKAEAYAQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKNAVDTANNRGVVVVAAAGNSGSTGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGNSFYYGKGLINVQAASN

SEQ ID NO: 4 - protein, 381 aa, Bacillus pumilus
  REGION 1..381: note = misc_feature - Wild-type BsAprE
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 5 - DNA, 32 bp, synthetic construct
  misc_feature 1..32: note = Synthetic
  source 1..32: mol_type = other DNA
aaaaaaactagtttagttagaagccgcttgaa

SEQ ID NO: 6 - DNA, 45 bp, synthetic construct
  misc_feature 1..45: note = Synthetic
  source 1..45: mol_type = other DNA
aaaaaatctagataaggaggtcaaaaatgtgcgtgaaaaagaaaa

SEQ ID NO: 7 - DNA, 20 bp, synthetic construct
  misc_feature 1..20: note = Synthetic
  source 1..20: mol_type = other DNA
ttagttagaagccgcttgaa

SEQ ID NO: 8 - DNA, 33 bp, synthetic construct
  misc_feature 1..33: note = Synthetic
  source 1..33: mol_type = other DNA
aaaaaagacgtcggagttctgagaattggtatg

SEQ ID NO: 9 - DNA, 36 bp, synthetic construct
  misc_feature 1..36: note = Synthetic
  source 1..36: mol_type = other DNA
aaaaaagaattcactacatttattgtacaacacgag

SEQ ID NO: 10 - DNA, 21 bp, synthetic construct
  misc_feature 1..21: note = Synthetic
  source 1..21: mol_type = other DNA
ggagttctgagaattggtatg

SEQ ID NO: 11 - DNA, 24 bp, synthetic construct
  misc_feature 1..24: note = Synthetic
  source 1..24: mol_type = other DNA
actacatttattgtacaacacgag

SEQ ID NO: 12 - DNA, 47 bp, synthetic construct
  misc_feature 1..47: note = Synthetic
  source 1..47: mol_type = other DNA
aaaaaatctagataaggaggtcaaaaatgagaagcaaaaaattgtgg

SEQ ID NO: 13 - DNA, 33 bp, synthetic construct
  misc_feature 1..33: note = Synthetic
  source 1..33: mol_type = other DNA
aaaaaaactagtttattgtgcagctgcttgtac

SEQ ID NO: 14 - DNA, 21 bp, synthetic construct
  misc_feature 1..21: note = Synthetic
  source 1..21: mol_type = other DNA
gagcggatacatatttgaatg

SEQ ID NO: 15 - DNA, 20 bp, synthetic construct
  misc_feature 1..20: note = Synthetic
  source 1..20: mol_type = other DNA
ggataacaatttcacacagg

SEQ ID NO: 16 - DNA, 36 bp, synthetic construct
  misc_feature 1..36: note = Synthetic
  source 1..36: mol_type = other DNA
gctgttatcgacacaggaattgactcttctcatcct

SEQ ID NO: 17 - DNA, 36 bp, synthetic construct
  misc_feature 1..36: note = Synthetic
  source 1..36: mol_type = other DNA
agagtcaattcctgtgtcgataacagctacttttac

SEQ ID NO: 18 - DNA, 36 bp, synthetic construct
  misc_feature 1..36: note = Synthetic
  source 1..36: mol_type = other DNA
taccaggacggccaatctcacggtacgcatgtagcc

SEQ ID NO: 19 - DNA, 36 bp, synthetic construct
  misc_feature 1..36: note = Synthetic
  source 1..36: mol_type = other DNA
cgtaccgtgagattggccgtcctggtatgggtttgt

SEQ ID NO: 20 - DNA, 36 bp, synthetic construct
  misc_feature 1..36: note = Synthetic
  source 1..36: mol_type = other DNA
gtgcttgattcatatggaagcggccaatatagctgg

SEQ ID NO: 21 - DNA, 36 bp, synthetic construct
  misc_feature 1..36: note = Synthetic
  source 1..36: mol_type = other DNA
ttggccgcttccatatgaatcaagcacttttactgc

SEQ ID NO: 22 - DNA, 36 bp, synthetic construct
  misc_feature 1..36: note = Synthetic
  source 1..36: mol_type = other DNA
gattcaacaggagacggccaatatagctggattatt

SEQ ID NO: 23 - DNA, 36 bp, synthetic construct
  misc_feature 1..36: note = Synthetic
  source 1..36: mol_type = other DNA
gctatattggccgtctcctgttgaatcaagcacttt

SEQ ID NO: 24 - DNA, 36 bp, synthetic construct
  misc_feature 1..36: note = Synthetic
  source 1..36: mol_type = other DNA
cttggcggacctaacggttctacagcgctgaaaaca

SEQ ID NO: 25 - DNA, 36 bp, synthetic construct
  misc_feature 1..36: note = Synthetic
  source 1..36: mol_type = other DNA
cgctgtagaaccgttaggtccgccaaggctcatgtt

SEQ ID NO: 26 - DNA, 36 bp, synthetic construct
  misc_feature 1..36: note = Synthetic
  source 1..36: mol_type = other DNA
gcagccggaaactcaggttcatccggaagcacaagc

SEQ ID NO: 27 - DNA, 36 bp, synthetic construct
  misc_feature 1..36: note = Synthetic
  source 1..36: mol_type = other DNA
tccggatgaacctgagtttccggctgcggcagcaac

SEQ ID NO: 28 - DNA, 36 bp, synthetic construct
  misc_feature 1..36: note = Synthetic
  source 1..36: mol_type = other DNA
caaagagcttcatcatccagcgcaggttctgagctt

SEQ ID NO: 29 - DNA, 36 bp, synthetic construct
  misc_feature 1..36: note = Synthetic
  source 1..36: mol_type = other DNA
acctgcgctggatgatgaagctctttggttgctgct

SEQ ID NO: 30 - DNA, 36 bp, synthetic construct
  misc_feature 1..36: note = Synthetic
  source 1..36: mol_type = other DNA
gctgtccttgatagcggaatccacgccgcacaccct

SEQ ID NO: 31 - DNA, 36 bp, synthetic construct
  misc_feature 1..36: note = Synthetic
  source 1..36: mol_type = other DNA
ggcgtggattccgctatcaaggacagctactttgac

SEQ ID NO: 32 - DNA, 36 bp, synthetic construct
  misc_feature 1..36: note = Synthetic
  source 1..36: mol_type = other DNA
acacaagacttttcatcacacggaactcacgtagcc

SEQ ID NO: 33 - DNA, 36 bp, synthetic construct
  misc_feature 1..36: note = Synthetic
  source 1..36: mol_type = other DNA
agttccgtgtgatgaaaagtcttgtgtggcatttgg

SEQ ID NO: 34 - DNA, 36 bp, synthetic construct
  misc_feature 1..36: note = Synthetic
  source 1..36: mol_type = other DNA
gtattagatcgtacaggcgatggacaatacagctgg

SEQ ID NO: 35 - DNA, 36 bp, synthetic construct
  misc_feature 1..36: note = Synthetic
  source 1..36: mol_type = other DNA
ttgtccatcgcctgtacgatctaatactttaacggc

SEQ ID NO: 36 - DNA, 36 bp, synthetic construct
  misc_feature 1..36: note = Synthetic
  source 1..36: mol_type = other DNA
gatcgttacggcagcggacaatacagctggattatc

SEQ ID NO: 37 - DNA, 36 bp, synthetic construct
  misc_feature 1..36: note = Synthetic
  source 1..36: mol_type = other DNA
gctgtattgtccgctgccgtaacgatctaatacttt

SEQ ID NO: 38 - DNA, 36 bp, synthetic construct
  misc_feature 1..36: note = Synthetic
  source 1..36: mol_type = other DNA
ttaggcggaccaacaggctccacagcgcttaaaaat

SEQ ID NO: 39 - DNA, 36 bp, synthetic construct
  misc_feature 1..36: note = Synthetic
  source 1..36: mol_type = other DNA
cgctgtggagcctgttggtccgcctaagctcatatt

SEQ ID NO: 40 - DNA, 36 bp, synthetic construct
  misc_feature 1..36: note = Synthetic
  source 1..36: mol_type = other DNA
gcagcagggaatgaaggttcaactggctctactagc

SEQ ID NO: 41 - DNA, 36 bp, synthetic construct
  misc_feature 1..36: note = Synthetic
  source 1..36: mol_type = other DNA
gccagttgaaccttcattccctgctgcggcaacgac

SEQ ID NO: 42 - DNA, 36 bp, synthetic construct
  misc_feature 1..36: note = Synthetic
  source 1..36: mol_type = other DNA
gtcagaaactcgttttctagcgcaggtcctgaatta

SEQ ID NO: 43 - DNA, 36 bp, synthetic construct
  misc_feature 1..36: note = Synthetic
  source 1..36: mol_type = other DNA
acctgcgctagaaaacgagtttctgacattgttgct

SEQ ID NO: 44 - DNA, 36 bp, synthetic construct
  misc_feature 1..36: note = Synthetic
  source 1..36: mol_type = other DNA
agcttaggcggagcaaacggctccacagcgcttaaa

SEQ ID NO: 45 - DNA, 36 bp, synthetic construct
  misc_feature 1..36: note = Synthetic
  source 1..36: mol_type = other DNA
tgtggagccgtttgctccgcctaagctcatattgat

SEQ ID NO: 46 - DNA, 36 bp, synthetic construct
  misc_feature 1..36: note = Synthetic
  source 1..36: mol_type = other DNA
agcttaggcggagcaacaggctccacagcgcttaaa

SEQ ID NO: 47 - DNA, 36 bp, synthetic construct
  misc_feature 1..36: note = Synthetic
  source 1..36: mol_type = other DNA
tgtggagcctgttgctccgcctaagctcatattgat

SEQ ID NO: 48 - DNA, 36 bp, synthetic construct
  misc_feature 1..36: note = Synthetic
  source 1..36: mol_type = other DNA
aattcaggttcatttggctctactagcaccgttggc

SEQ ID NO: 49 - DNA, 36 bp, synthetic construct
  misc_feature 1..36: note = Synthetic
  source 1..36: mol_type = other DNA
gctagtagagccaaatgaacctgaattccctgctgc

SEQ ID NO: 50 - DNA, 36 bp, synthetic construct
  misc_feature 1..36: note = Synthetic
  source 1..36: mol_type = other DNA
tcaactggctcttttagcaccgttggctatcctgca

SEQ ID NO: 51 - DNA, 36 bp, synthetic construct
  misc_feature 1..36: note = Synthetic
  source 1..36: mol_type = other DNA
gccaacggtgctaaaagagccagttgaacctgaatt

SEQ ID NO: 52 - DNA, 36 bp, synthetic construct
  misc_feature 1..36: note = Synthetic
  source 1..36: mol_type = other DNA
aaagtattagattcatacggcagcggacaatacagc

SEQ ID NO: 53 - DNA, 36 bp, synthetic construct
  misc_feature 1..36: note = Synthetic
  source 1..36: mol_type = other DNA
tccgctgccgtatgaatctaatactttaacggcata

SEQ ID NO: 54 - DNA, 36 bp, synthetic construct
  misc_feature 1..36: note = Synthetic
  source 1..36: mol_type = other DNA
gtattagatcgttttggcagcggacaatacagctgg

SEQ ID NO: 55 - DNA, 36 bp, synthetic construct
  misc_feature 1..36: note = Synthetic
  source 1..36: mol_type = other DNA
ttgtccgctgccaaaacgatctaatactttaacggc

SEQ ID NO: 56 - DNA, 36 bp, synthetic construct
  misc_feature 1..36: note = Synthetic
  source 1..36: mol_type = other DNA
gtattagatcgttggggcagcggacaatacagctgg

SEQ ID NO: 57 - DNA, 36 bp, synthetic construct
  misc_feature 1..36: note = Synthetic
  source 1..36: mol_type = other DNA
ttgtccgctgccccaacgatctaatactttaacggc

SEQ ID NO: 58 - DNA, 36 bp, synthetic construct
  misc_feature 1..36: note = Synthetic
  source 1..36: mol_type = other DNA
agcagtggatatgcatcttatacgggaacatcgatg

SEQ ID NO: 59 - DNA, 36 bp, synthetic construct
  misc_feature 1..36: note = Synthetic
  source 1..36: mol_type = other DNA
tcccgtataagatgcatatccactgcttggtactgt

SEQ ID NO: 60 - DNA, 36 bp, synthetic construct
  misc_feature 1..36: note = Synthetic
  source 1..36: mol_type = other DNA
agcagtggatatggctcttatacgggaacatcgatg

SEQ ID NO: 61 - DNA, 36 bp, synthetic construct
  misc_feature 1..36: note = Synthetic
  source 1..36: mol_type = other DNA
tcccgtataagagccatatccactgcttggtactgt

SEQ ID NO: 62 - DNA, 36 bp, synthetic construct
  misc_feature 1..36: note = Synthetic
  source 1..36: mol_type = other DNA
tacggcagcggatcatacagctggattatcagcggt

SEQ ID NO: 63 - DNA, 36 bp, synthetic construct
  misc_feature 1..36: note = Synthetic
  source 1..36: mol_type = other DNA
aatccagctgtatgatccgctgccgtaacgatctaa

SEQ ID NO: 64 - DNA, 36 bp, synthetic construct
  misc_feature 1..36: note = Synthetic
  source 1..36: mol_type = other DNA
tacggcagcggatattacagctggattatcagcggt

SEQ ID NO: 65 - DNA, 36 bp, synthetic construct
  misc_feature 1..36: note = Synthetic
  source 1..36: mol_type = other DNA
aatccagctgtaatatccgctgccgtaacgatctaa

SEQ ID NO: 66 - DNA, 36 bp, synthetic construct
  misc_feature 1..36: note = Synthetic
  source 1..36: mol_type = other DNA
ttaggcggaccatcaggctccacagcgcttaaaaat

SEQ ID NO: 67 - DNA, 36 bp, synthetic construct
  misc_feature 1..36: note = Synthetic
  source 1..36: mol_type = other DNA
cgctgtggagcctgatggtccgcctaagctcatatt

SEQ ID NO: 68 - DNA, 36 bp, synthetic construct
  misc_feature 1..36: note = Synthetic
  source 1..36: mol_type = other DNA
ggcagcggacaatttagctggattatcagcggtatt

SEQ ID NO: 69 - DNA, 36 bp, synthetic construct
  misc_feature 1..36: note = Synthetic
  source 1..36: mol_type = other DNA
gataatccagctaaattgtccgctgccgtaacgatc

SEQ ID NO: 70 - DNA, 36 bp, synthetic construct
  misc_feature 1..36: note = Synthetic
  source 1..36: mol_type = other DNA
ggcagcggacaatggagctggattatcagcggtatt

SEQ ID NO: 71 - DNA, 36 bp, synthetic construct
  misc_feature 1..36: note = Synthetic
  source 1..36: mol_type = other DNA
gataatccagctccattgtccgctgccgtaacgatc

SEQ ID NO: 72 - DNA, 36 bp, synthetic construct
  misc_feature 1..36: note = Synthetic
  source 1..36: mol_type = other DNA
agcttaggcggatcaacaggctccacagcgcttaaa

SEQ ID NO: 73 - DNA, 36 bp, synthetic construct
  misc_feature 1..36: note = Synthetic
  source 1..36: mol_type = other DNA
tgtggagcctgttgatccgcctaagctcatattgat

SEQ ID NO: 74 - DNA, 36 bp, synthetic construct
  misc_feature 1..36: note = Synthetic
  source 1..36: mol_type = other DNA
tatacatcttattcaggaacatcgatggcatctcct

SEQ ID NO: 75 - DNA, 36 bp, synthetic construct
  misc_feature 1..36: note = Synthetic
  source 1..36: mol_type = other DNA
catcgatgttcctgaataagatgtatatccactgct

SEQ ID NO: 76 - DNA, 36 bp, synthetic construct
  misc_feature 1..36: note = Synthetic
  source 1..36: mol_type = other DNA
ggatatacatcttttacgggaacatcgatggcatct

SEQ ID NO: 77 - DNA, 36 bp, synthetic construct
  misc_feature 1..36: note = Synthetic
  source 1..36: mol_type = other DNA
cgatgttcccgtaaaagatgtatatccactgcttgg

SEQ ID NO: 78 - DNA, 36 bp, synthetic construct
  misc_feature 1..36: note = Synthetic
  source 1..36: mol_type = other DNA
ggatatacatctctgacgggaacatcgatggcatct

SEQ ID NO: 79 - DNA, 36 bp, synthetic construct
  misc_feature 1..36: note = Synthetic
  source 1..36: mol_type = other DNA
cgatgttcccgtcagagatgtatatccactgcttgg

SEQ ID NO: 80 - DNA, 36 bp, synthetic construct
  misc_feature 1..36: note = Synthetic
  source 1..36: mol_type = other DNA
tacggcgcttatacaggaacgtccatggcgactcct

SEQ ID NO: 81 - DNA, 36 bp, synthetic construct
  misc_feature 1..36: note = Synthetic
  source 1..36: mol_type = other DNA
catggacgttcctgtataagcgccgtaagtgcctcc

SEQ ID NO: 82 - DNA, 36 bp, synthetic construct
  misc_feature 1..36: note = Synthetic
  source 1..36: mol_type = other DNA
tatacatcttataatggaacatcgatggcatctcct

SEQ ID NO: 83 - DNA, 36 bp, synthetic construct
  misc_feature 1..36: note = Synthetic
  source 1..36: mol_type = other DNA
catcgatgttccattataagatgtatatccactgct

SEQ ID NO: 84 - DNA, 36 bp, synthetic construct; identical to SEQ ID NO: 80
  misc_feature 1..36: note = Synthetic
  source 1..36: mol_type = other DNA

SEQ ID NO: 85 - DNA, 36 bp, synthetic construct; identical to SEQ ID NO: 81
  misc_feature 1..36: note = Synthetic
  source 1..36: mol_type = other DNA

SEQ ID NO: 86 - DNA, 36 bp, synthetic construct; identical to SEQ ID NO: 82
  misc_feature 1..36: note = Synthetic
  source 1..36: mol_type = other DNA

SEQ ID NO: 87 - DNA, 36 bp, synthetic construct; identical to SEQ ID NO: 83
  misc_feature 1..36: note = Synthetic
  source 1..36: mol_type = other DNA

SEQ ID NO: 88 - DNA, 36 bp, synthetic construct
  misc_feature 1..36: note = Synthetic
  source 1..36: mol_type = other DNA
aactcaggttcaacaggaagcacaagcacagtcggc

SEQ ID NO: 89 - DNA, 36 bp, synthetic construct
  misc_feature 1..36: note = Synthetic
  source 1..36: mol_type = other DNA
gcttgtgcttcctgttgaacctgagtttccggctgc

SEQ ID NO: 90 - DNA, 36 bp, synthetic construct
  misc_feature 1..36: note = Synthetic
  source 1..36: mol_type = other DNA
ggaggcacttacacagcttataacggaacgtccatg

SEQ ID NO: 91 - DNA, 36 bp, synthetic construct
  misc_feature 1..36: note = Synthetic
  source 1..36: mol_type = other DNA
tccgttataagctgtgtaagtgcctccaggaagtgt

SEQ ID NO: 92 - DNA, 36 bp, synthetic construct
  misc_feature 1..36: note = Synthetic
  source 1..36: mol_type = other DNA
aaagtgcttgatagatatggaagcggccaatatagc

SEQ ID NO: 93 - DNA, 36 bp, synthetic construct
  misc_feature 1..36: note = Synthetic
  source 1..36: mol_type = other DNA
gccgcttccatatctatcaagcacttttactgcata

SEQ ID NO: 94 - DNA, 36 bp, synthetic construct
  misc_feature 1..36: note = Synthetic
  source 1..36: mol_type = other DNA
aattcaggttcatcaggctctactagcaccgttggc

SEQ ID NO: 95 - DNA, 36 bp, synthetic construct
  misc_feature 1..36: note = Synthetic
  source 1..36: mol_type = other DNA
gctagtagagcctgatgaacctgaattccctgctgc

SEQ ID NO: 96 - DNA, 36 bp, synthetic construct
  misc_feature 1..36: note = Synthetic
  source 1..36: mol_type = other DNA
agcaaccaaagaaattcattctccagcgcaggttct

SEQ ID NO: 97 - DNA, 36 bp, synthetic construct
  misc_feature 1..36: note = Synthetic
  source 1..36: mol_type = other DNA
gctggagaatgaatttctttggttgctgctgtttac